(19) United States
(12) Patent Application Publication
(10) Pub. No.: US 2016/0008387 A9
(43) Pub. Date: Jan. 14, 2016

(54) AGENTS AND DEVICES FOR AFFECTING NERVE FUNCTION

(71) Applicant: Northwind Medical, Inc., San Jose, CA (US)

(72) Inventors: Emily A. Stein, San Leandro, CA (US); Christina D. Swanson, Cambridge, MA (US); Michael A. Evans, Palo Alto, CA (US); Kondapavulur T. Venkateswara-Rao, San Jose, CA (US)

(21) Appl. No.: 14/399,512

(22) PCT Filed: May 7, 2013
(86) PCT No.: PCT/US2013/039904

§ 371 Date: Nov. 7, 2014

Related U.S. Application Data
(63) Continuation-in-part of application No. 14/395,485, filed on Oct. 18, 2014.
Application No. 14/395,485 is a continuation-in-part of application No. 13/096,446, filed on Apr. 28, 2011, now Pat. No. 9,056,184.
Application No. 13/096,446 is a continuation of application No. 13/014,700, filed on Jan. 26, 2011, now Pat. No. 8,975,233.
Application No. 14/395,485 is a continuation-in-part of application No. 13/014,702, filed on Jan. 26, 2011, now abandoned.
(60) Provisional application No. 61/644,134, filed on May 8, 2012.
(60) Provisional application No. 61/551,921, filed on Oct. 26, 2011.
(60) Provisional application No. 61/336,838, filed on Jan. 26, 2010.
(60) Provisional application No. 61/336,838, filed on Jan. 26, 2010.

(51) Int. Cl. A61K 31/7048; A61M 5/32; A61K 31/401; A61K 45/06; A61K 31/4166; A61K 31/55; A61K 33/14; A61M 25/10; A61K 31/405

(52) U.S. Cl. CPC A61K 31/7048; A61M 25/10; A61M 5/3298; A61K 31/401; A61K 31/405; A61K 31/4166; A61K 31/55; A61K 33/14; A61K 45/06; A61M 2025/105; A61M 2025/1079; A61M 2025/1081

(57) ABSTRACT

Agents and devices for affecting nerve function are described. In some variations, a combination of agents, e.g., a cardiac glycoside, an ACE inhibitor, and an NSAID are delivered to affect nerve function. The agent may be delivered locally in a site-specific manner to a targeted nerve or portion of a nerve. For example, the agent may be delivered locally to the renal nerves to impair their function and treat hypertension. One variation of a delivery device includes one or more needle housings supported by a balloon. A delivery needle is slidably disposed within a needle lumen of each needle housing.

### CROSS-REFERENCE TO RELATED APPLICATIONS

This application claims the benefit of U.S. provisional patent application No. 61/644,134, filed May 8, 2012. This application is also a continuation-in-part of U.S. patent application Ser. No. 13/014,700, filed Jan. 26, 2011, U.S. patent application Ser. No. 13/014,702, filed Jan. 26, 2011, and U.S. patent application Ser. No. 13/096,446, filed Apr. 28, 2011, which are nonprovisionals of U.S. patent application Ser. No. 61/336,838, filed Jan. 26, 2010, each of which are incorporated by reference in their entirety.

### BACKGROUND

It has been found that sympathetic nerve feedback from the kidneys is at least partially responsible for hypertension, and that denervating the renal nerves has the effect of lowering blood pressure. One method of renal denervation involves the use of radiofrequency (RF) energy to ablate the renal nerves. This method generally involves positioning a RF catheter inside the renal artery, and placing it in contact with the wall of the renal artery before RF energy is applied to the vascular tissue and renal nerves. Damage to the walls of the renal arteries and other surrounding tissue is one disadvantage of this approach. Furthermore, the long-term effects of RF ablation are not well understood. For example, the response of the body to tissue killed by RF ablation may cause an undesirable necrosis or “dirty” response, versus an apoptosis response, which is a programmed, quiet cell death that triggers a phagocyte cleanup. Lastly, the destruction of the renal nerves by RF ablation is not a well-controlled (an all-or-none) process, and does not readily lend itself to adjustment in terms of specifically targeting nerve cells and limiting the damage caused to neighboring cells.

Another method of renal denervation involves the use of agents such as guanethidine or botulinum toxin to denervate the renal nerves. When this method is used, a delivery catheter is typically positioned inside the renal artery, and a needle is passed through the wall of the renal artery before the guanethidine or botulinum toxin is injected in or around the renal nerves. However, these agents affect nerve function by acting at the synapses of sympathetic nerves. Because the renal nerves are made up of long nerve cells which begin at or near the spinal cord, or at or near the renal plexus near the aortic ostia of renal arteries, and terminate inside the kidneys, accessing the synapses well inside the kidneys makes local delivery difficult. This requires the delivery of agents over extended distances inside the body, and increases the likelihood of the agents entering the systemic circulation and exposing renal tissue, surrounding tissue, and the kidneys to these agents that may have undesirable effects.

Accordingly, it would be beneficial to have compositions that include one or more agents that affect the function of nerves, but which reduce the likelihood of damage to surrounding tissues, e.g., vascular and renal tissues. For example, nerve affecting agents that impair the function of the renal nerves while reducing the likelihood of damage to the renal arteries and other tissues in its vicinity would be useful.

It would also be beneficial to have compositions including one or more nerve affecting agents that are capable of permanently preventing neuronal signal transmission and insulating the kidney from sympathetic electrical activity to and from the kidney over long periods of time. Agents and agent compositions that can be titrated to control the amount of nerve function that is affected would be useful. Nerve affecting compositions that are effective in small volumes and low concentrations acting on a portion of the nerve or nerve cell would also be useful.

Peripheral nerves are known for their remarkable ability to regenerate after injury in contrast to nerves in the central nervous system. It is therefore desirable to have agents and compositions that have a prolonged and permanent affect on nerve function by preventing the regrowth or regeneration of neuronal cells.

Furthermore, it would be useful to have devices which can deliver these agents locally in small volumes to nerves and nerve cells in a targeted, site-specific manner, so as to reduce damage to surrounding tissues and reduce the side effects associated with systemic administration.

### SUMMARY

Described here are nerve affecting compositions for the treatment of various medical conditions and methods and devices for locally delivering the compositions proximate the nerves. The nerve affecting compositions may be used to treat medical conditions such as, but not limited to, hypertension, diabetes, atrial fibrillation, sleep apnea, heart failure, chronic kidney disease, fibromyalgia, obesity, dementia, and depression. Specifically, compositions that affect the function of renal nerves are described. The renal nerve affecting compositions may be delivered to any suitable tissue near or adjacent the renal nerves. When the compositions are delivered proximate or adjacent the renal nerves, they may be delivered to any suitable tissue or layer or tissue, e.g., the adventitial layer of the vascular wall. In some instances, the compositions that affect renal nerve function are delivered extravascularly, i.e., outside the blood vessel wall.

The nerve affecting compositions may include one or more nerve affecting agents. In one variation, the nerve affecting composition includes a single agent. In other variations, the nerve affecting composition includes at least two agents. In yet further variations, the nerve affecting compositions include at least three agents. Surprisingly, it has been found that the use of certain combinations of agents allows the concentration of the agents within the formulation to be lowered compared to use of a single agent, while still achieving a desired efficacy. Specifically, when a cardiac glycoside such as digoxin is combined with one or more additional agents, the effect on nerve function may be enhanced. Digoxin has inotropic properties, and in excess quantities is known to be cardiotoxic. However, as further described below, it was surprising to find that digoxin in combination with other agents could affect nerve (e.g., renal nerve) function.

A plurality of nerve affecting agents may be combined to form a single composition, or each nerve affecting agent may be separately delivered to the target nerve simultaneously or sequentially. Exemplary nerve affecting agents include without limitation, cardiac glycosides, calcium channel blockers, sodium channel blockers, potassium channel blockers, angiotensin-converting enzyme (ACE) inhibitors, antibiotics, excitatory amino acids, and nonsteroidal anti-inflammatory drugs (NSAIDS), alpha-adrenergic blockers, beta-adrenergic blockers, benzodiazepines, nitroglycerin, amyl nitrate, pentaerythritol tetranitrate, and magnesium sulfate.

Methods for treating hypertension in a patient are also described. The methods generally comprise locally delivering a composition to a portion of a renal nerve in an amount that affects function of the renal nerve and lowers blood pressure of the patient, wherein the composition comprises a cardiac glycoside, an ACE inhibitor, and an NSAID.

Also described are methods for treating a disease condition of the autonomic nervous system in a patient. The methods may comprise delivering a nerve affecting composition to a portion of a targeted nerve locally in an amount that affects function of the targeted nerve and alleviates one or more symptoms of the disease condition in the patient.

### BRIEF DESCRIPTION OF THE DRAWINGS

FIG. 1A shows a nerve cell 100 of the peripheral nervous system.

FIG. 1B shows an enlarged view of the axon 130.

FIG. 1C shows an enlarged view of a synapse 300.

FIGS. 2A-2E show how a voltage potential is maintained across the cell membrane 150 by a sodium-potassium pump 210.

FIGS. 3A-3E show how an action potential is propagated along the axon 130 by the sodium channels 220 and the potassium channels 230.

FIGS. 4A-4D show how a neural signal is propagated across a synapse 300.

FIG. 5 shows how a cardiac glycoside may affect nerve function.

FIG. 6 shows how a calcium channel blocker may affect nerve function.

FIG. 7 shows how a sodium channel blocker may affect nerve function.

FIG. 8 shows how an angiotensin-converting enzyme (ACE) inhibitor may affect nerve function.

FIG. 9 shows how an antibiotic may affect nerve function.

FIG. 10 shows how an excess amount of an excitatory amino acid may affect nerve function.

FIG. 11 shows how a non-steroidal anti-inflammatory drug (NSAID) affect nerve function.

FIGS. 12A-12D show the results of several different agents on rat sciatic nerves.

FIGS. 13A-13B show histologies from the hind leg of a rat injected with digoxin at 72 hours and 30 days.

FIGS. 14A-14G show one variation of a delivery catheter 400.

FIGS. 15A-15D show one variation of a method for using delivery catheter 400.

FIGS. 16A-16H show another variation of a delivery device 500.

FIGS. 17A-17D show a method for using delivery device 500 according to another variation.

FIGS. 18A-18E show yet another variation of a delivery device 600.

FIGS. 19A-19E show one variation of a method for using delivery device 600.

FIG. 20 is a bar graph that compares the degree of conductive block affected by an exemplary agent used alone at a high dose with its use in combination with other agents, where the dose of the agents in the combination is low in a rat sciatic nerve block model.

FIG. 21 is a bar graph that compares the amount of sensory block affected by exemplary agents used alone and in combination in a rat sciatic nerve block model.

FIG. 22 is a bar graph that compares the degree of conductive block affected by exemplary agents used alone at a high dose with their combination at low doses in a rat sciatic nerve block model.

### DETAILED DESCRIPTION

Described here are nerve affecting compositions for the treatment of various medical conditions and methods and devices for locally delivering the compositions proximate the nerves in human patients. The nerve affecting compositions may be used to treat medical conditions such as, but not limited to, hypertension, diabetes, atrial fibrillation, sleep apnea, heart failure, chronic kidney disease, fibromyalgia, obesity, dementia, and depression. Specifically, compositions that affect the function of renal nerves are described. The compositions may include one or more nerve affecting agents that can either permanently prevent neuronal signal transmission, or which can be titrated to control the amount of nerve function that is affected. In some variations, compositions that include a combination of nerve affecting agents may be beneficial. The nerve affecting compositions are generally delivered locally in small volumes proximate the nerves, e.g., the renal nerves, in a site-specific manner. This may be accomplished using endovascular catheters with expandable structures configured to include slidable needles for advancement to the target area and delivery of the agent(s).

### I. GENERAL PHYSIOLOGY OF NERVE CONDUCTION

The sympathetic nervous system represents one of the electrical conduction systems of the body. With age and disease, this electrical conduction system degenerates.

The degeneration of the sympathetic nervous system is often accompanied by inflammation, expressed as overactivity of signal transmission or firing by the nerve cells. The agents, devices, and methods described herein may generally seek to affect the function of nerve cells by reducing or impairing this overactivity to treat a wide range of attendant disease conditions such as hypertension, diabetes, atrial fibrillation, sleep apnea, heart failure, chronic kidney disease, fibromyalgia, obesity, dementia, and depression, as stated above, and many others.

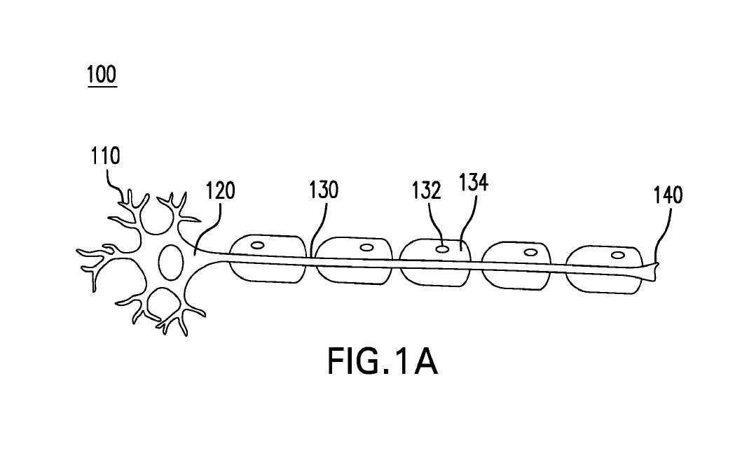

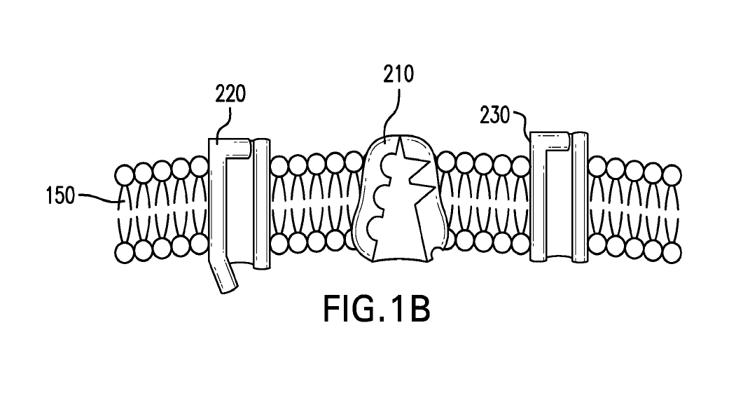

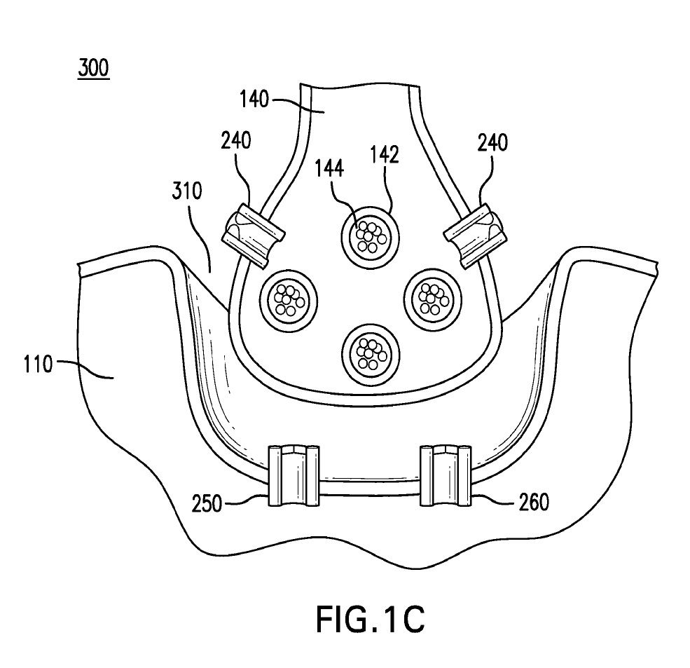

FIG. 1A shows a nerve cell 100 of the peripheral nervous system. The nerve cell 100 includes dendrites 110, a body 120, and an axon 130. The branches of the dendrites 110 receive neural signals from other nerve cells and converge at the body 120. From the body 120, the axon 130 extends away and ends in axon terminals 140. An axon terminal 140 transmits neural signals to a dendrite of another nerve cell.

A nerve bundle is made up of a multiple of nerve cells. The individual nerve cells in a nerve bundle can perform different functions, depending on how the nerve cell is terminated. These functions include sensory, motor, pressure, and other functions.

The renal nerves may include nerve cells having axons of about 5 to about 25 cm or more in length, extending from the spinal cord to the kidney.

FIG. 1B shows an enlarged view of the axon 130, showing a cell membrane 150. The cell membrane 150 is embedded with sodium-potassium pumps 210, sodium channels 220, and potassium channels 230. The sodium-potassium pumps 210 maintain a voltage potential across the cell membrane 150. The sodium channels 220 and the potassium channels 230 propagate an action potential along the axon 130.

FIG. 1C shows an enlarged view of a synapse 300. An axon terminal 140 of a presynaptic nerve cell and a dendrite 110 of a postsynaptic nerve cell are separated by a synaptic cleft 310. The axon terminal 140 includes calcium channels 240 embedded in the cell membrane 150. The axon terminal also includes vesicles 142 containing neurotransmitters 144. The dendrite 110 of the postsynaptic nerve cell includes ligand-gated sodium channels 250 and ligand-gated calcium channels 260 which are activated by the neurotransmitters 144.

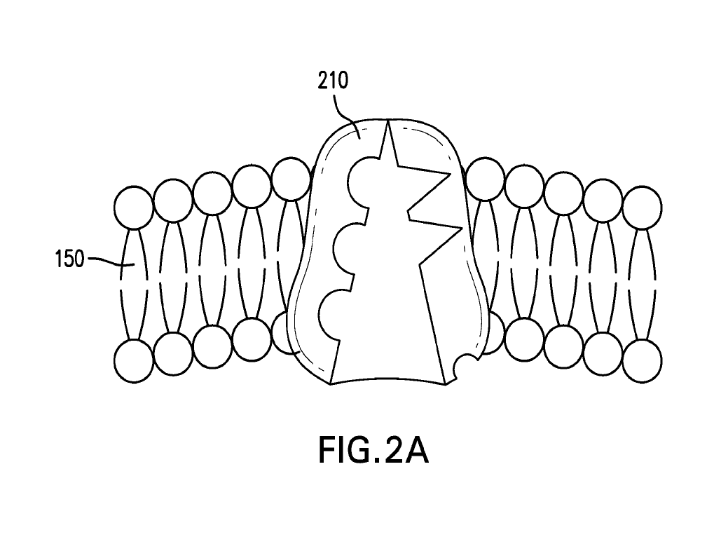

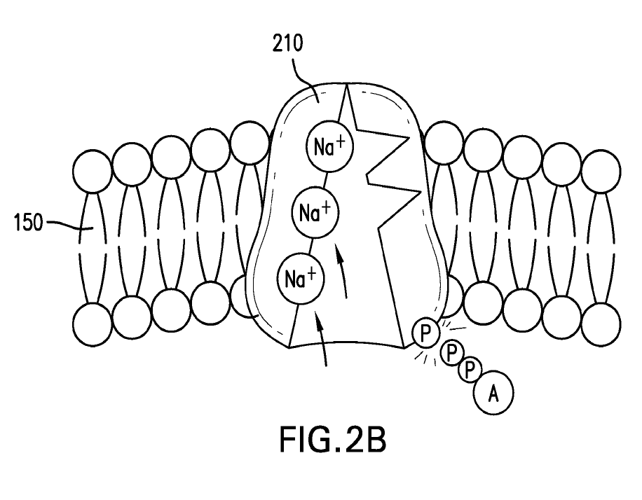

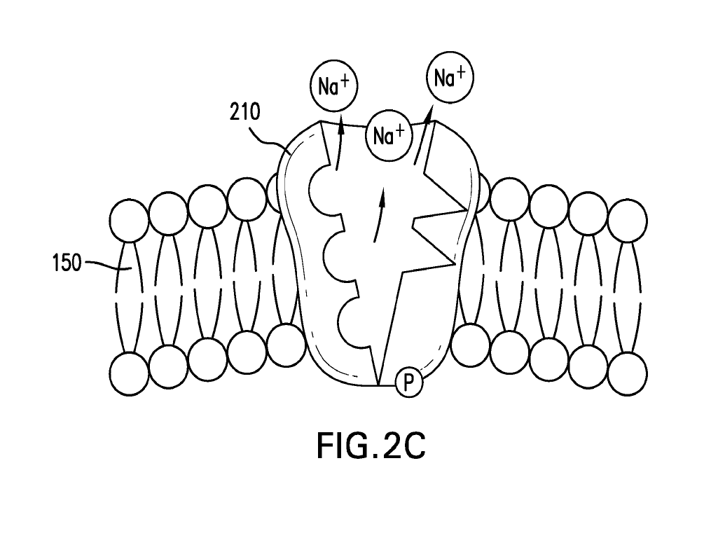

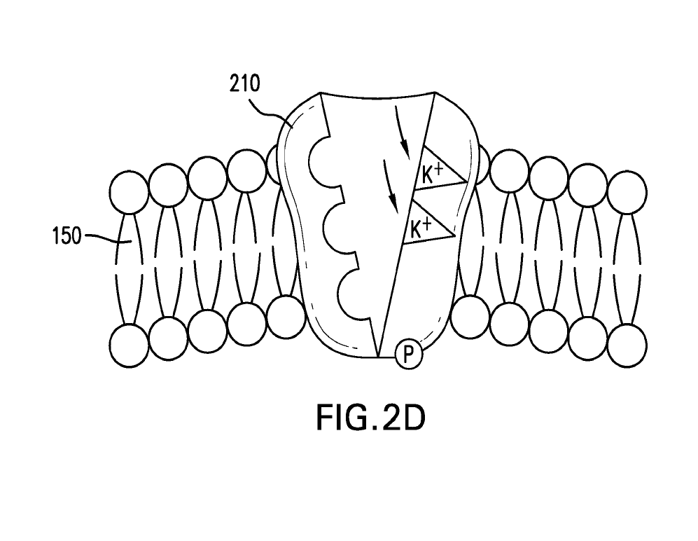

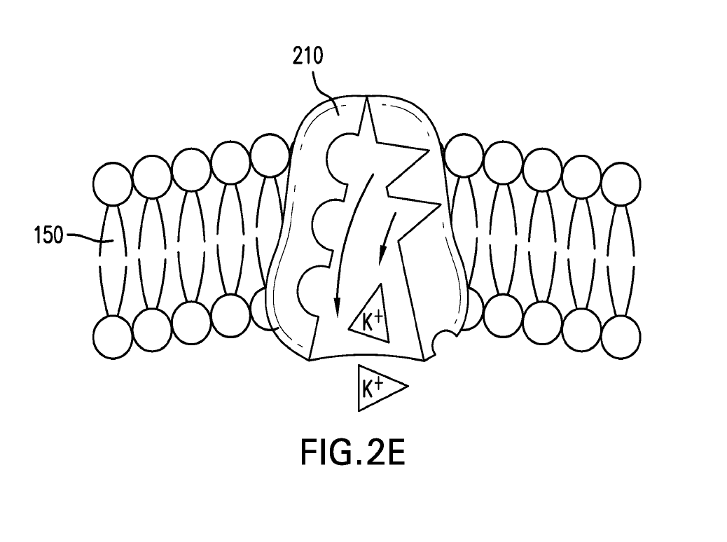

FIGS. 2A-2E show how a voltage potential is maintained across the cell membrane 150 by a sodium-potassium pump (Na+/K+-ATPase) 210. FIG. 2A shows a sodium-potassium pump 210 embedded in the cell membrane 150. FIG. 2B shows sodium ions (Na+) and an ATP molecule binding to the sodium-potassium pump 210 on the inside of the cell membrane 150. FIG. 2C shows the adenosine triphosphate (ATP) molecule being broken down into adenosine diphosphate (ADP), and the sodium-potassium pump 210 changing shape and transporting the sodium ions (Na+) to the outside of the cell membrane 150. FIG. 2D shows potassium ions (K+) binding to the sodium-potassium pump 210 on the outside of the cell membrane 150. FIG. 2E shows the phosphate molecule being released, and the sodium-potassium pump 210 reverting to its original shape and transporting the potassium ions (K+) to the inside of the cell membrane 150.

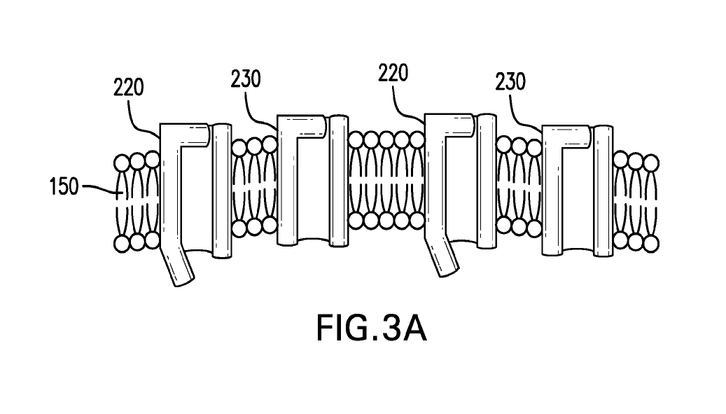

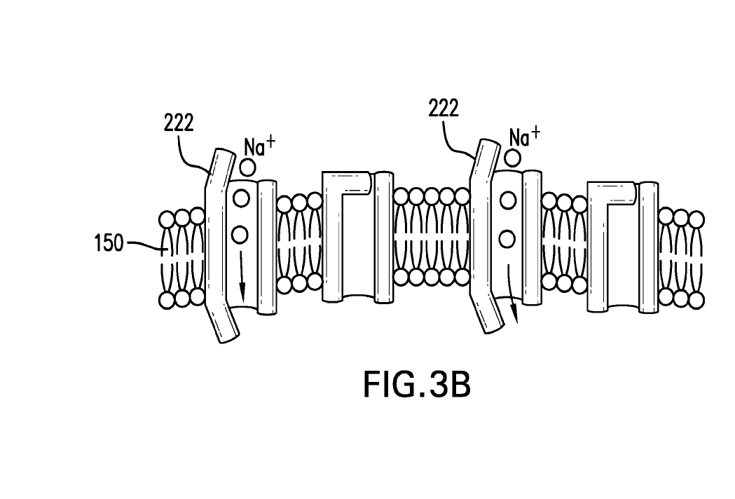

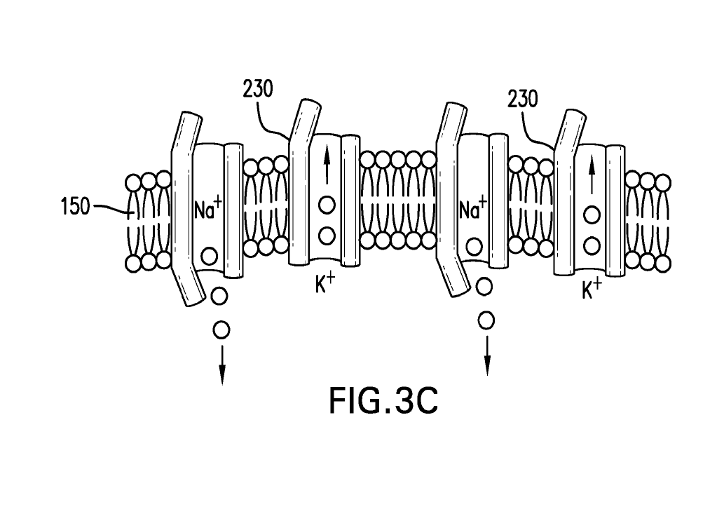

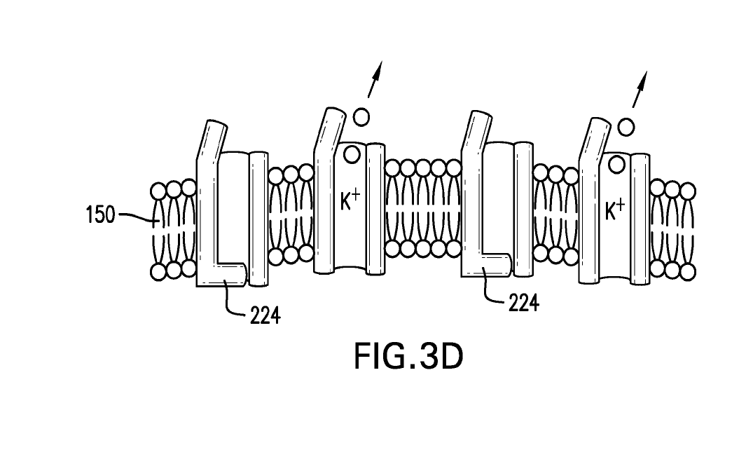

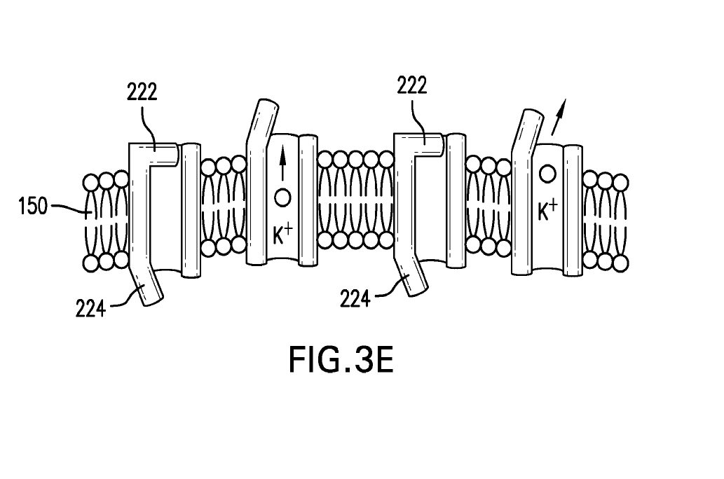

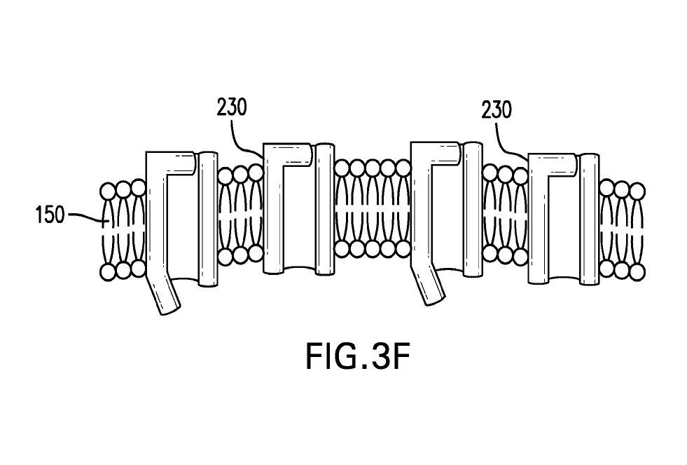

FIGS. 3A-3E show how an action potential is propagated along the axon 130 by the sodium channels 220 and the potassium channels 230. FIG. 3A shows sodium channels 220 and potassium channels 230 embedded in the cell membrane 150. FIG. 3B shows the arrival of an action potential, which opens activation gates 222 of the sodium channels 220, allowing the diffusion of sodium ions (Na+) into the inside of the cell membrane 150. FIG. 3C shows the action potential also opening the potassium channels 230, allowing the diffusion of potassium ions (K+) to the outside of the cell membrane 150. The combined effect of this is to depolarize the cell membrane 150, which propagates the action potential along the axon 130. FIG. 3D shows the inactivation gates 224 of the sodium channels 220 closed. FIG. 3E shows the activation gates 222 of the sodium channels 220 closed, and the inactivation gates 224 open. FIG. 3F shows the potassium channels 230 closed.

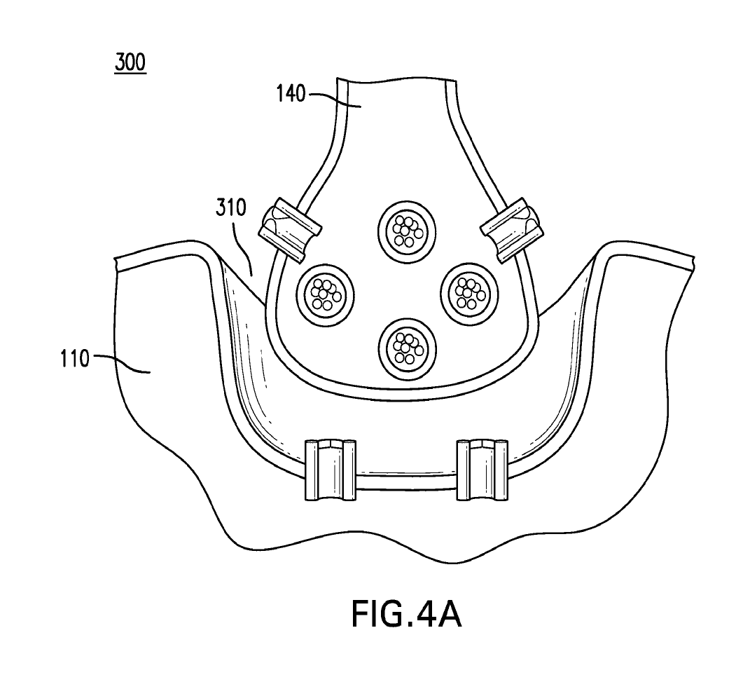

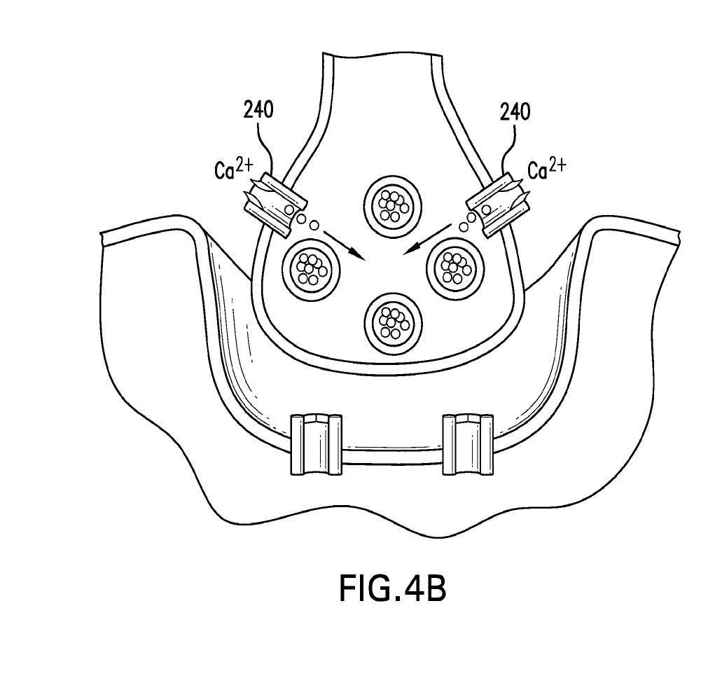

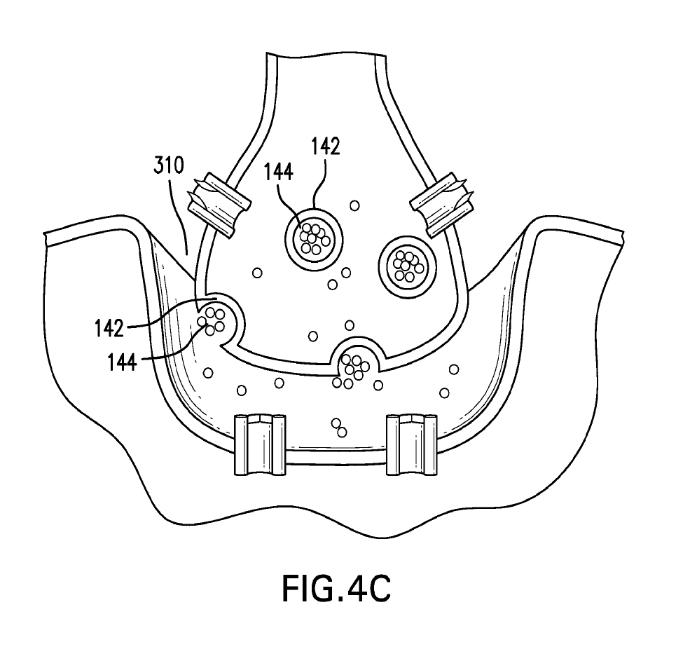

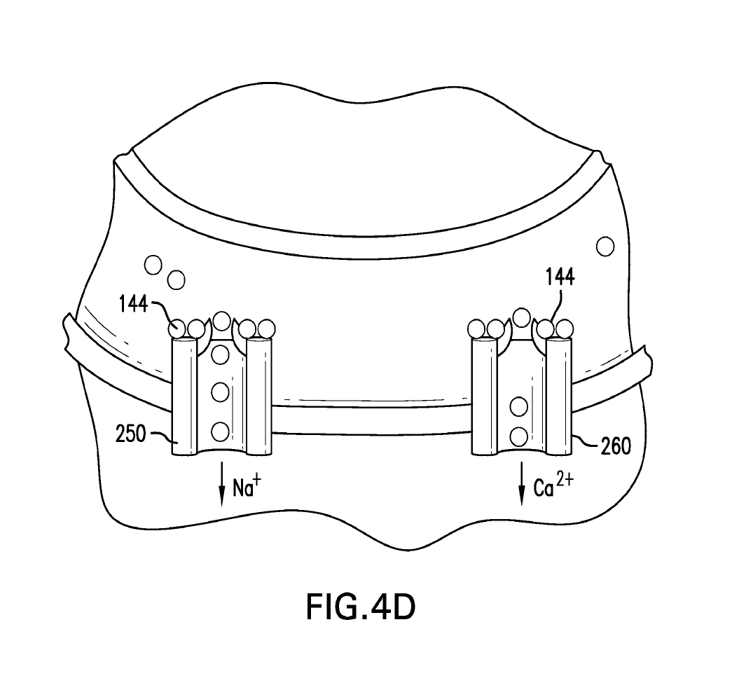

FIGS. 4A-4D show how a neural signal is propagated across a synapse 300. FIG. 4A shows an axon terminal 140 of a presynaptic nerve cell and a dendrite 110 of a postsynaptic nerve cell separated by the synaptic cleft 310. FIG. 4B shows the arrival of an action potential, which opens the calcium channels 240 and allows the diffusion of calcium ions (Ca2+) into the inside of the cell membrane 150. FIG. 4C shows the vesicles 142 releasing the neurotransmitters 144 into the synaptic cleft 310. FIG. 4D shows the neurotransmitters 144 binding to the ligand-gated sodium channels 250 and ligand-gated calcium channels 260, which opens them and allows the diffusion of sodium ions (Na+) and calcium ions (Ca2+) into the dendrite 110 to produce an action potential in the postsynaptic nerve cell.

Referring back to FIG. 1A, the axon 130 is surrounded by Schwann cells 132 which produce a myelin sheath 134 which covers the axon 130. The myelin sheath 134 is an insulator which serves to increase the speed of propagation of the action potential along the axon 130.

### II. NERVE AFFECTING COMPOSITIONS

The nerve affecting compositions described herein may include a single agent or a combination of agents that affect nerve function. When a combination of agents are employed, two, three, or more than three agents may be used. The nerve affecting compositions may affect nerve function, e.g., renal nerve function, by mechanisms such as inducing apoptosis of nerve cells, blocking propagation or conduction of an action potential and/or blocking repolarization of the nerve cell membrane, and inducing nerve cell death. In some variations, the nerve affecting compositions induce apoptosis of nerve cells. In other variations, the nerve affecting compositions permanently affect nerve cell function. In yet further variations, the nerve affecting compositions temporarily (e.g., reversibly) affect nerve cell function. In one variation, the nerve affecting composition affects renal nerve function.

Nerves receive signals, react to signals and send signals. Many signals are received and processed simultaneously and involve multiple pathways. A single agent may act to modulate a signaling pathway upstream, within or downstream from a nerve cell. The use of certain additional agents may have an incremental, additive or synergistic effect depending, e.g., on the role(s) played by the molecular targets. Additionally, the administration of an agent can act in a synergistic manner when used in combination with a second agent whereby first and second agents target different molecules involved in different nerve cell functions. For example, using a beta-blocker to block reception of upstream activating signals in combination with an ion channel blocker to block membrane potentials may inhibit: (i) ligand-receptor complex formation, (ii) receptor-mediated endocytosis of bound ligand, (iii) intracellular signaling, (iv) nerve cell action potential, (v) nerve cell repolarization, (vi) release of nerve signaling products, and (vii) downstream activation of neighboring nerves. Accordingly, affecting nerve function in the ways previously stated may result in increased effectiveness, increased durability or a combination of both with respect to nerve blockade.

Another example of synergy may occur when administering two or more blocking agents that target transporters with affinity for different ions. In this example, a calcium channel blocker, a chloride transporter blocker and a sodium/potassium transporter blocker may inhibit the transport of different ions. The effect of the disruption of ion homeostasis may result in a significant and prolonged impairment in nerve function, which may eventually lead to nerve cell death.

Another example of synergy can occur when administering two or more blocking agents that target both non-nerve cells and nerve cells. In this example, the first agent may target a nerve cell and the second agent may target a cell upstream or downstream of a nerve cell (e.g., Schwann cells, immune cells, adipocytes, kidney cells, and/or smooth muscle cells).

Another example of synergy can occur when administering two or more agents that target the afferent and efferent nerve bundles or afferent and efferent fibers within the same nerve bundle. Compositions of agents can be administered to achieve efferent-specific effects. Other compositions of agents can be administered to achieve afferent-specific effects.

Yet another example of synergy can occur when administering two or more agents that affect nerve function over a period of time. In this example, the first agent acts immediately to block the signal transmission between neurons, disruption of ion homeostasis and eventually lead to cell death. The second agent prevents axon regeneration by blocking non-neuronal cells in the release of extracellular matrix components, cytokines and growth factors that can support axon regrowth.

Agent combinations may provide a synergistic effect on the target nerve, as previously stated. That is, the degree of nerve function affected may be enhanced when a combination of agents are used in comparison to when an agent is used alone. Synergism can be the result of more than one agent altering the same signaling pathway in a neuron. Synergism can also be the result of the use of different or separately selected agents to target different signaling pathways in a neuron. Synergism can further be the result of using agents that target signaling pathways upstream of a neuron and also within a neuron. For example, a first agent may be used that prevents firing (release of neurotransmitters, polarization, and/or opening of channels) of the nerve cells and a second agent that prevents repolarization may also be delivered. In a second example, a first agent and a second agent may be used wherein the first agent prevents a certain signal from being produced in a nerve cell, and the second agent interrupts ion homeostasis in a neuron to prevent uptake of the released signal to produce an enhanced effect on nerve function.

### III. NERVE AFFECTING AGENTS

Exemplary agents that may be used in the nerve affecting compositions described herein include without limitation, cardiac glycosides, calcium channel blockers, sodium channel blockers, potassium channel blockers, angiotensin-converting enzyme (ACE) inhibitors, antibiotics, excitatory amino acids, and nonsteroidal anti-inflammatory drugs (NSAIDS), alpha-adrenergic blockers, beta-adrenergic blockers, benzodiazepines, nitroglycerin, amyl nitrate, pentaerythritol tetranitrate, and magnesium sulfate. One or more of these agents can be combined in the nerve affecting compositions, as further described below. These agents and classes of agents may act through different mechanisms.

Exemplary cardiac glycosides that may be employed include without limitation, digoxin, proscillaridin, ouabain, digitoxin, bufalin, cymarin, oleandrin, and combinations thereof. In some variations, it may be useful to include digoxin as the cardiac glycoside in the nerve affecting compositions described herein. Digoxin is FDA-approved, comes in injectable formulations, and is available as a generic. The pharmacokinetic and pharmacodynamic properties of digoxin are desirable for affecting nerve function. Digoxin is extremely hydrophobic and the high lipid content surrounding nerves and nerve bundles allows digoxin to penetrate the outer lipid-rich sheath. Digoxin has a half-life of 36-48 hours in healthy individuals and is excreted by the kidneys, which reduce the risk of diffusion-related effects on sites outside of the zone of administration. Other cardiac glycosides with lipophilic profiles include bufalin, ouabain, and others.

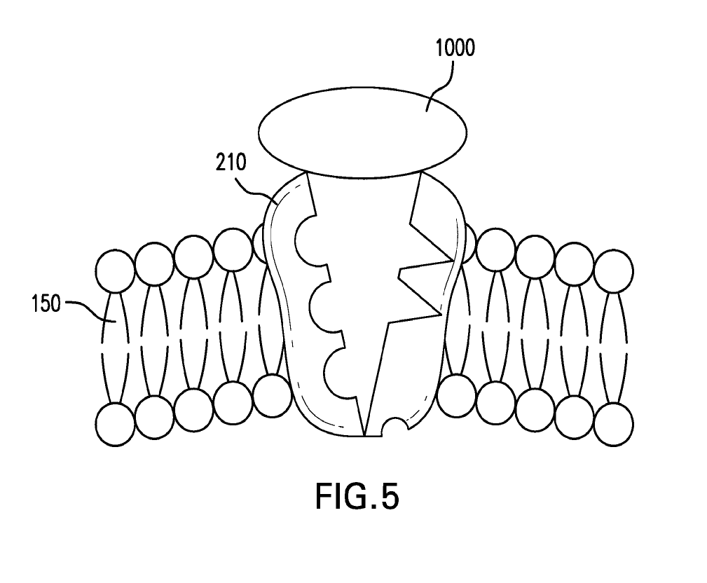

FIG. 5 shows how a cardiac glycoside may affect nerve function. Cardiac glycosides target sodium-potassium pumps 210. A cardiac glycoside molecule 1000 binds to the extracellular surface of a sodium-potassium pump 210. This inhibits the sodium-potassium pump 210, which reduces the transport of sodium ions out of the nerve cell 100. This increases the sodium ion concentration inside the nerve cell 100, which leads to apoptosis and impairs nerve function. Cardiac glycosides may also bind to organic anion transporters (OATs), which inhibits other membrane transport processes and leads to apoptosis.

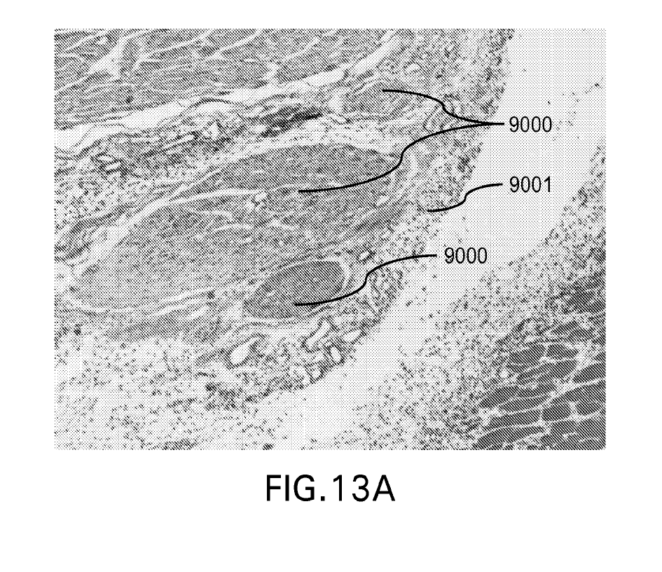

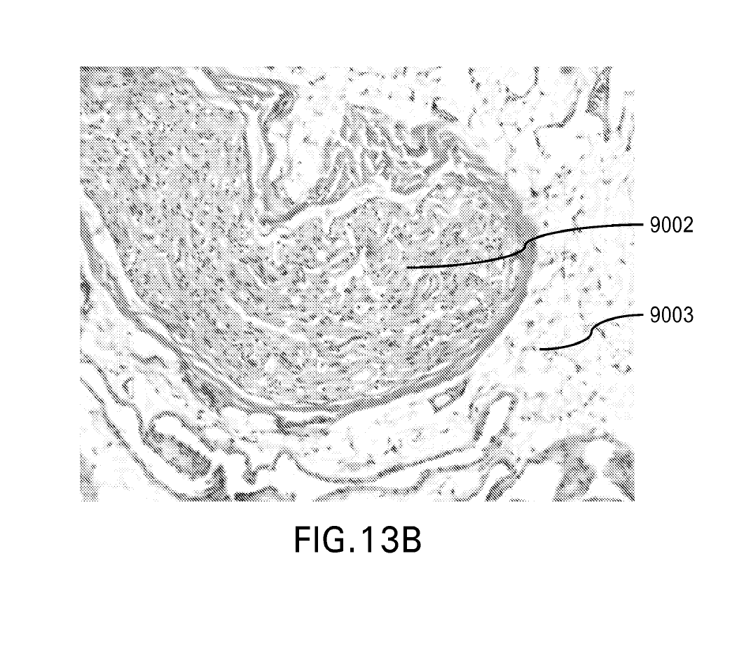

Cardiac glycosides may be delivered to a nerve in a targeted, site-specific manner, such as with the delivery devices described below and in FIGS. 13A-18F. They may target sodium-potassium pump along the long axonal segment of the nerve cell. This allows for a highly targeted and localized, site-specific effect by cardiac glycosides on a single nerve cell or a nerve cell bundle. This also allows for the use of very small volumes of agent to be delivered to a small, targeted area. Digoxin and other cardiac glycosides may be administered to a nerve, e.g., a renal nerve, in volumes of about 0.01 cc to about 1.5 cc, about 0.01 cc to about 0.5 cc, or about 0.05 cc to about 0.2 cc, in a single injection. Targeted delivery also allows the use of lower doses than when administered systemically, an advantage given the narrow therapeutic index of cardiac glycosides. This also avoids toxicity to other cells, given the amounts necessary to induce apoptosis, and given that many other types of cells other than nerve cells are also contain sodium-potassium pumps 210. In some instances, it may be beneficial to administer digoxin and other cardiac glycosides so that tissue concentration of the agent is about 0.1 mg to about 5.0 mg per gram of tissue, about 0.25 mg to about 3.0 mg per gram of tissue, or about 0.5 to about 2.0 mg per gram of tissue. Targeted delivery also avoids the need for the agents to be transported over large distances to reach the synaptic cleft, which may inhibit the transmission of catecholamines between neurons, as is the case with guanethidine, or the need to ablate large volumes of surrounding tissue to ablate nerves, as may happen with RF ablation.

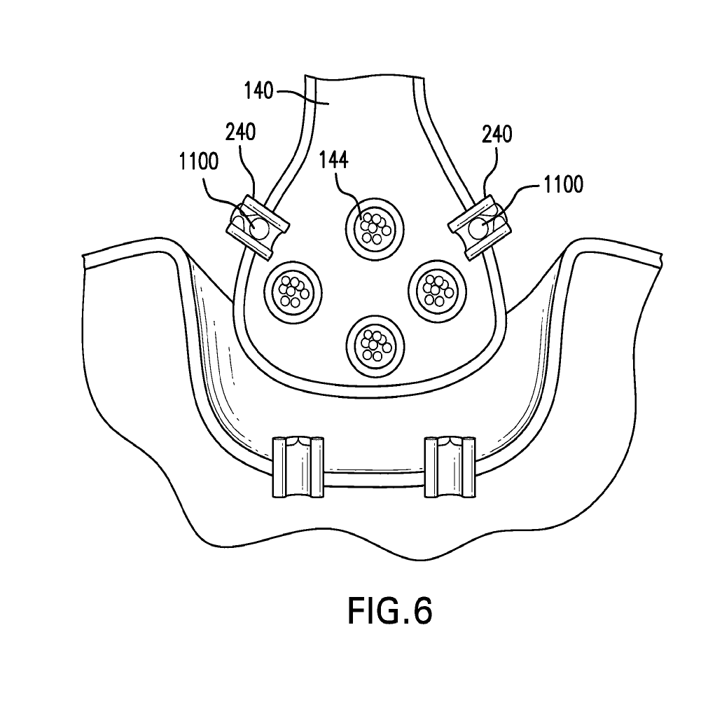

Exemplary calcium channel blockers that may be used are selected from the group consisting of, but not limited to, amlodipine, aranidipine, azelnidipine, cilnidipine, felodipine, and combinations thereof. FIG. 6 shows how a calcium channel blocker may affect nerve function. Calcium channel blockers target calcium channels 240. A calcium channel blocker molecule 1100 binds to any one of several sites in a calcium channel 240, depending on the specific calcium channel blocker. This blocks the calcium channel 240, which inhibits the diffusion of calcium ions into the nerve cell 100 when an action potential is received. The lower calcium ion concentration inside the nerve cell 100 reduces the ability of the axon terminal 140 to release neurotransmitters 144 at the synapse 300, and thus impairs nerve function. Calcium channel blockers include amlodipine, aranidipine, azelnidipine, cilnidipine, felodipine and others.

Calcium channel blockers may be delivered to a nerve in a targeted, site-specific manner, such as with the delivery devices described below and in FIGS. 13A-18F. This allows the use of lower doses than when administered systemically. This also avoids impairing the function of cells other than the targeted nerve cells, given that many other types of cells other than nerve cells are also rich in calcium channels 240.

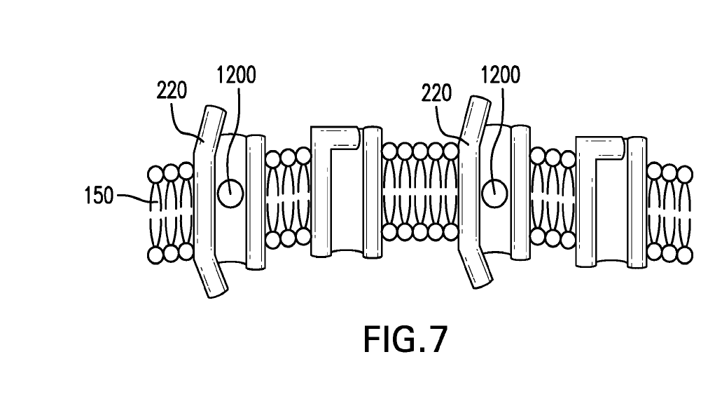

Exemplary sodium channel blockers that may be used include, but are not limited to, phenytoin, lithium chloride, carbamazepine, and combinations thereof. FIG. 7 shows how a sodium channel blocker may affect nerve function. Sodium channel blockers target sodium channels 220. A sodium channel blocker molecule 1200 binds to any one of several sites in a sodium channel 220, depending on the specific sodium channel blocker. This blocks the sodium channel 220, which inhibits the diffusion of sodium ions into the nerve cell 100 when an action potential is received. This inhibits the nerve from propagating action potentials and impairs nerve function. This effect is useful to inhibit high-frequency repetitive firing of action potentials caused by excessive stimulation.

Sodium channel blockers may be delivered to a nerve in a targeted, site-specific manner, such as with the delivery devices described below and in FIGS. 13A-18F. This allows for delivery of low volumes of agent in small concentrations to the axonal segments of nerve cells, and effectively impairs nerve function with minimal damage to surrounding tissue or organs and limits the risk of the agents entering the systemic circulation. This also allows the use of lower doses than when administered systemically. This also avoids impairing the function of cells other than the targeted nerve cells, given that many other types of cells other than nerve cells are also rich in sodium channels 220.

ACE-inhibitors that may be included in the nerve affecting compositions include, but are not limited to, captopril, enalapril, lisinopril, ramipril, and combinations thereof. In one variation, captopril may be used. Captopril is FDA-approved, is available as a generic, has a streamlined synthesis, comes in injectable formulations, has a well-established safety profile, and has a well-established dosing regimen. Captopril is excreted by the kidneys with a short half-life of 1.9 hours.

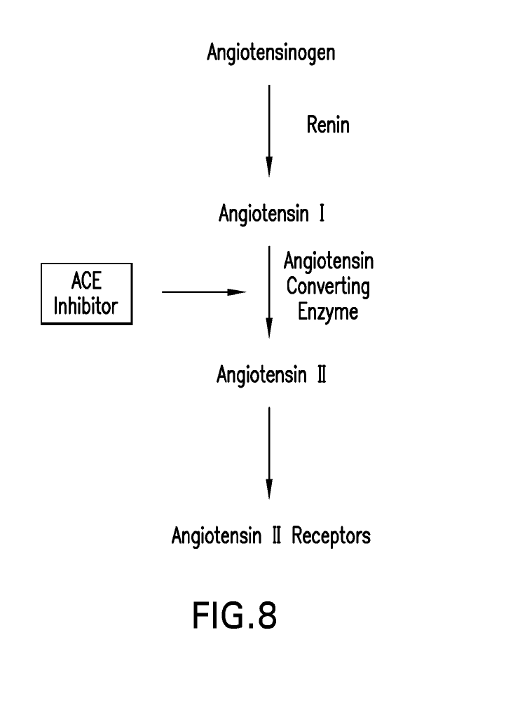

FIG. 8 shows how an angiotensin-converting enzyme (ACE) inhibitor may affect nerve function. ACE inhibitors target angiotensin-converting enzymes, disrupting the renin-angiotensin cycle. An ACE inhibitor inhibits ACE, which converts angiotensin I to angiotensin II, a more biologically active substrate for many cells including sympathetic nerves. ACE inhibition decreases angiotensin II production and thereby reduces nerve-specific production of norepinepherine. Blocking ACE by an ACE inhibitor not only reduces sympathetic nerve activity, it also decreases aldosterone release by the adrenal cortex. The combined effects result in the lowering of arteriolar resistance and renovascular resistance leading to increased excretion of sodium in the urine (natriuresis). ACE inhibitors include captopril, enalapril, lisinopril, ramipril, and others.

ACE inhibitors may be delivered to a nerve in a targeted, site-specific manner, such as with the delivery devices described below and in FIGS. 13A-18F. Site-specific administration of ACE inhibitors results in decreased local peripheral nerve activity.

The antibiotics that may be used include without limitation, metronidazole, fluoroquinolones (such as ciprofloxacin, levofloxacin, moxifloxacin and others), chloramphenicol, chloriquine, clioquinol, dapsone, ethambutol, griseofulvin, isoniazid, linezolid, mefloquine, nitrofurantoin, podophyllin resin, suramin, and combinations thereof.

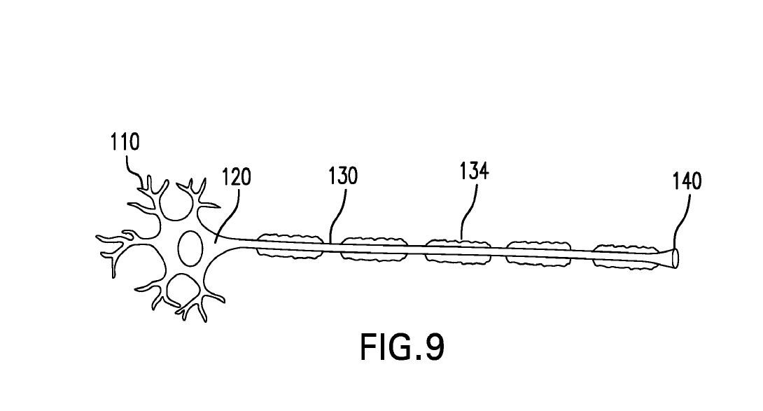

FIG. 9 shows how an antibiotic may affect nerve function. Antibiotics may cause RNA and thiamine antagonism. Antibiotics may also cause demyelination of the nerve cells, which interferes with the ability of the nerve cells to conduct signals. The quinolone and fluoroquinolone classes of antibiotics have been shown to cause irreversible peripheral neuropathy. Antibiotics include metronidozole, fluoroquinolones (such as ciprofloxacin, levofloxacin, moxifloxacin and others), chloramphenicol, chloriquine, clioquinol, dapsone, ethambutol, griseofulvin, isoniazid, linezolid, mefloquine, nitrofurantoin, podophyllin resin, suramin, and others.

Antibiotics may be delivered to a nerve in a targeted, site-specific manner, such as with the delivery devices described below and in FIGS. 13A-18F. This allows the use of lower doses than when administered systemically, an advantage given the effects of some of these antibiotics on the central nervous system. This also minimizes damage to other tissue in the vicinity of the targeted nerve.

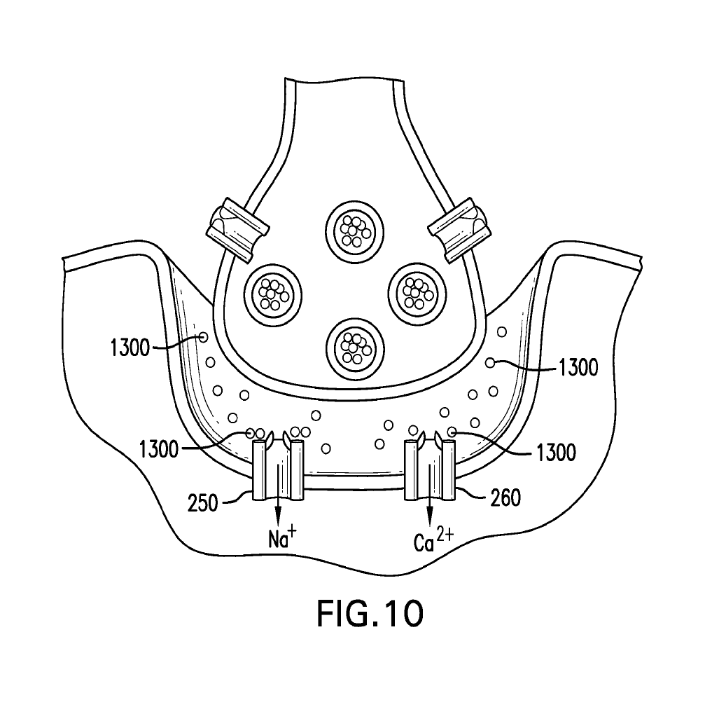

Excitatory amino acids that may be used are selected for the group consisting of, but not limited to, monosodium glutamate, domoic acid, and combinations thereof. FIG. 10 shows how an excess amount of an excitatory amino acid may affect nerve function. Excitatory amino acids target neurotransmitter receptors in the postsynaptic nerve cell. An excess amount of an excitatory amino acid 1300 overactivates the neurotransmitter receptors of the sodium channels 250 and calcium channels 260, which leads to the uptake of high amounts of sodium and calcium ions in the postsynaptic nerve cell. These high sodium and calcium ion concentrations lead to destruction of cell components, apoptosis, and impaired nerve function. Excitatory amino acids include monosodium glutamate, domoic acid and others.

Excess amounts of excitatory amino acids may be delivered to a nerve in a targeted, site-specific manner, such as with the delivery devices described below and in FIGS. 13A-18F. This allows the use of lower doses than when administered systemically. This also avoids impairing the function of cells other than nerve cells, given that many other types of cells other than nerve cells are also rich in calcium channels 240.

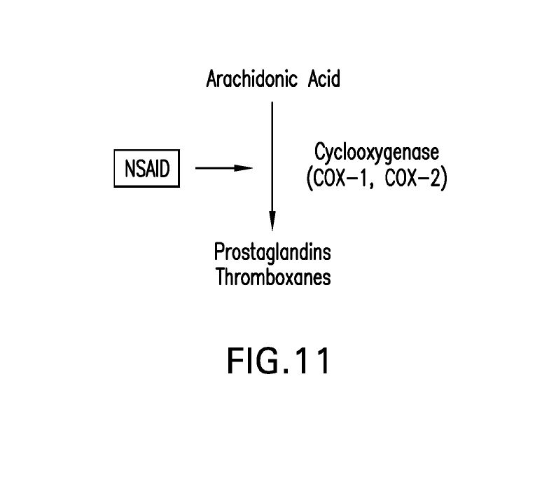

Exemplary NSAIDS that may be employed include without limitation, indomethacin, aspirin, ibuprofen, naproxen, celecoxib, and combinations thereof. In one variation, indomethacin is used. Indomethacin is FDA-approved, comes in injectable formulations, and is available as a generic. Indomethacin has a half-life of 4.5 hours and the majority of the agent is excreted by the kidneys. FIG. 11 shows how a non-steroidal anti-inflammatory drug (NSAID) may affect nerve function. NSAIDs target the cyclooxygenase (COX) enzyme. An NSAID blocks the COX-1 and COX-2 enzymes, which suppresses production of prostaglandins and thromboxanes and reduces synaptic signaling. Additionally, a subclass of prostaglandins are involved in healing and the administration of prostaglandin E2 enhances healing. Like other analgesics, NSAIDs can act in various ways on the peripheral and central nervous systems. NSAIDs include indomethacin, aspirin, ibuprofen, naproxen, celecoxib, and others.

NSAIDs may be delivered to a nerve in a targeted, site-specific manner, such as with the delivery devices described below and in FIGS. 13A-18F. This is advantageous over systemic administration because of adverse drug reactions (ADRs) to NSAIDs in the kidneys. Blocking prostaglandin production in the kidneys is undesirable, as prostaglandins are essential in maintaining normal glomerular perfusion and glomerular filtration rate.

Local delivery of agents to affect nerve function may not be permanent, lasting from a few months to a few years. The sympathetic nervous system may return to its degenerated, overactive condition as the nerve cells regrow and transmit signals to and from the kidneys. If an extended effect is desired, agents may be included that may prevent nerve cell regrowth locally without causing detrimental effects to the central nervous system or surrounding tissue to permanently impair or affect nerve function and prevent nerve overactivity. These agents include a variety of nerve growth inhibitors, which may be used in a time-release formulation. Other nerve affecting agents that may be used in the compositions described herein include small molecule inhibitors, kinase inhibitors, neutralizing or blocking antibodies, myelin-derived molecules, extracellular matrix components, and neurotrophic factors.

Nerve growth inhibitors prevent regrowth of the nerve after nerve cell injury or nerve cell death. Nerve growth inhibitors may prolong the effect on nerve function from months to years, or even make permanent the effect on nerve function.

A nerve growth inhibitor may be a single agent, or include two or more agents. A nerve growth inhibitor may include a small molecule inhibitor, a kinase inhibitor, a neutralizing or blocking antibody, a myelin-derived molecule, a sulfate proteoglycan, and/or extracellular matrix components.

Small molecule inhibitors may include, but are not limited to, cyclic-adenosine analogs and molecules targeting enzymes including Arginase I, Chondroitinase ABC, β-secretase BACE1, urokinase-type plasminogen activator, and tissue-type plasminogen activator. Inhibitors of arginase include, but are not limited to, N-hydroxy-L-arginine and 2(S)-amino-6-boronohexonic acid. β-secretase inhibitors include, but are not limited to, N-Benzyloxycarbonyl-Val-Leu-leucinal, H-Glu-Val-Asn-Statine-Val-Ala-Glu-Phe-NH₂, H-Lys-Thr-Glu-Glu-Ile-Ser-Glu-Val-Asn-Stat-Val-Ala-Glu-Phe-OH. Inhibitors of urokinase-type and tissue-type plasminogen activators include, but are not limited to, serpin E1, Tiplaxtinin, and plasminogen activator inhibitor-2.

Kinase inhibitors may target, but are not limited to targeting, Protein Kinase A, PI 3 Kinase, ErbB receptors, Trk receptors, Jaks/STATs, and fibroblast growth factor receptors. Kinase inhibitors may include, but are not limited to, staurosporine, H 89 dihydrochloride, cAMPS-Rp, triethylammonium salt, KT 5720, wortmannin, LY294002, IC486068, IC87114, GDC-0941, Gefitinib, Erlotinib, Lapatinib, AZ623, K252a, KT-5555, Cyclotraxin-B, Lestaurtinib, Tofacitinib, Ruxolitinib, SB1518, CYT387, LY3009104, TG101348, WP-1034, PD173074, and SPRY4.

Neutralizing or blocking antibodies may target, but are not limited to targeting, kinases, enzymes, integrins, neuregulins, cyclin D1, CD44, galanin, dystroglycan, repulsive guidance molecule, neurotrophic factors, cytokines, and chemokines Targeted neurotrophic factors may include, but are not limited to, nerve growth factor, neurotrophin 3, brain-derived neurotrophic factor, and glial-cell-line derived neurotrophic factor. Targeted cytokines and chemokines may include, but are not limited to, interleukin-6, leukemia inhibitor factor, transforming growth factor β1, and monocyte-chemotactic protein 1.

Myelin-derived molecules may include, but are not limited to, myelin-associated glycoprotein, oligodendrocyte myelin glycoprotein, Nogo-A/B/C, Semaphorin 4D, Semaphorin 3A, and ephrin-B3.

Sulfate proteoglycans may include, but are not limited to, keratin sulfate proteoglycans and chondroitin sulfate proteoglycans such as neurocan, brevican, versican, phosphacan, aggrecan, and NG2.

Extracellular matrix components may include, but are not limited to, all known isoforms of laminin, fibrinogen, fibrin, and fibronectin.

Fibronectin binds to integrins such as alpha5beta1 on Schwann cells and neurons. Schwann cells adhere to fibronectin in order to migrate, and fibronectin acts as chemo-attractant and mitogen to these cells. Fibronectin aids the adhesion and outgrowth of regenerating axons. Agents which target fibronectin to impair nerve regrowth may thus include (1) isoforms of fibronectin that antagonize, rather than promote, integrin signaling, (2) blocking/neutralizing antibodies against certain fibronectin isoforms that promote integrin signaling, and/or (3) blocking/neutralizing antibodies that reduce fibronectin/integrin binding, integrin internalization or integrin grouping. One example of a humanized monoclonal antibody targeting fibronectin is Radretumab.

Laminins mediate the adhesion of neurons and Schwann cells to the extracellular matrix acting as a guide and “go” signal for regrowth. Laminin chains such as alpha2, alpha4, beta1 and gamma1 are upregulated following peripheral nerve injury and signal to neurons and Schwann cells through beta1 integrins such as alpha1beta1, alpha3beta1, alpha6beta1 and alpha7beta1 integrins. Agents which target laminins to impair nerve regrowth may thus include (1) antibodies that neutralize the effects of laminins, (2) laminin isoforms that antagonize rather than promote axon regrowth, and/or (3) blocking/neutralizing antibodies that reduce laminin/integrin binding, integrin internalization, or integrin grouping.

Collagen and fibrin promote nerve repair of a gap when added to the gap at low concentration, oriented in a longitudinal manner. However, fibrin (and perhaps collagen) may hinder nerve regeneration in some situations. First, unorganized fibrinogen in gel may retard nerve regeneration by confusing the growth pathways. Second, mice deficient in fibrinolytic enzymes such as tissue plasminogen activator or plasminogen have exacerbated injuries after sciatic nerve crush. This is believed to be due to fibrin deposition as fibrin depletion rescued the mice. In vitro experiments showed that fibrin downregulated Schwann cell myelin production and kept them in a proliferating, nonmyelinating state. Thus, at least a few different agents may be used to impair nerve regrowth. First, collagen or fibrinogen or the combination may be added at high concentration, in an unorganized state, via a gel injection at the site of injury. Second, small molecule inhibitors or neutralizing antibodies against tissue plasminogen activator or plasminogen may be used. Third, fibrin deposition may be mimicked by addition of peptides with the heterodimeric integrin receptor binding sequence arginine-glycin-asparagin.

Neurotrophic factors promote the growth of neurons. These include Nerve Growth Factor, Neurotrophin 3, Brain-derived neurotrophic factor. Agents which target neurotrophic factors to impair nerve regrowth may thus include neutralizing/blocking antibodies against neurotrophic factors or their respective receptors.

Glial growth factor (GGF) is produced by neurons during peripheral nerve regeneration, and stimulates the proliferation of Schwann cells. Agents which target GGF to impair nerve regrowth may thus include blocking/neutralizing antibodies against GGF.

Cyclic adenosine monophosphate (cAMP) is a second messenger that influences the growth state of the neuron. cAMP activates Protein Kinase A which induces the transcription of IL-6 and arginase I. Arginase I synthesizes polyamines which is considered one way that cAMP promotes neurite outgrowth. Knowledge of this pathway that promotes neurite outgrowth allows for identification of numerous targets for inhibiting neurite outgrowth. For instance, cAMP and Protein Kinase A may be targeted. Although the stereospecific cAMP phosphorothioate analog activates Protein Kinase A, other conformation such as the antagonistic Rp-cAMPs inhibit Protein Kinase A activity and may thus be used. Small molecules that inhibit Protein Kinase A or neutralizing/blocking antibodies that prevent cAMP from binding Protein Kinase A, or that prevent activation of Protein Kinase A via an alternative mechanism, may be used. Examples of inhibitors of Protein Kinase A include H 89 dihydrochloride, cAMPS-Rp, triethylammonium salt, and KT 5720. Further down the pathway, small molecule inhibitors of arginase I and polyamine synthesis may be used to reduce neurite outgrowth. Inhibitors of Arginase I may include but are not limited to, 2(S)-amino-6-boronohexonic acid and other boronic acid inhibitors.

Myelin-associated inhibitors are components of myelin expressed in the CNS by oligodendrocytes that impair neurite outgrowth in vitro and in vivo. Myelin-associated inhibitors include Nogo-A, myelin-associated glycoprotein (MAG), oligodendrocyte myelin glycoprotein (OMgp), ephrin-B3, and semaphorin 4D. NogoA, MAG and OMgp interact with Nogo-66 receptor 1 and the paired immunoglobulin-like receptor B to limit axon growth. Furthermore, transgenic expression of Nogo C, an isoform on Nogo A, in Schwann cells delays peripheral nerve regeneration. Any of these may be used to impair nerve regrowth.

Chondroitin sulfate proteoglycans (CSPGs) are upregulated by reactive astrocytes in the glial scar following nerve injury. They include neurocan, versican, brevican, phosphacan, aggrecan and NG2. Interfering with CSPG function is known to promote nerve growth in the CNS. Thus, CSPGs may be used to reduce nerve regrowth.

Non-myelin derived axon regeneration inhibitors are found in the CNS, but not derived from myelin. They include repulsive guidance molecule (RGM) and semaphorin 3A. Antibodies or small molecule inhibitors targeting these molecules promote functional recovery following spinal cord injury in rats. Thus, these molecules may be used to reduce nerve regrowth. Furthermore, these molecules activate Rho A which activates ROCK2 kinase, indicating that small molecules or antibodies that activate ROCK2 may be used to reduce neurite outgrowth. Examples of ROCK2 inhibitors include Fasudil hydrochloride which inhibits cyclic nucleotide dependent- and Rho-kinases, HA 1100 hydrochloride which is a cell-permeable, Rho-kinase inhibitor, dihydrochloride which is a selective Rho-kinase (ROCK) inhibitor, and dihydrochloride which is a selective inhibitor of isoform p160ROCK.

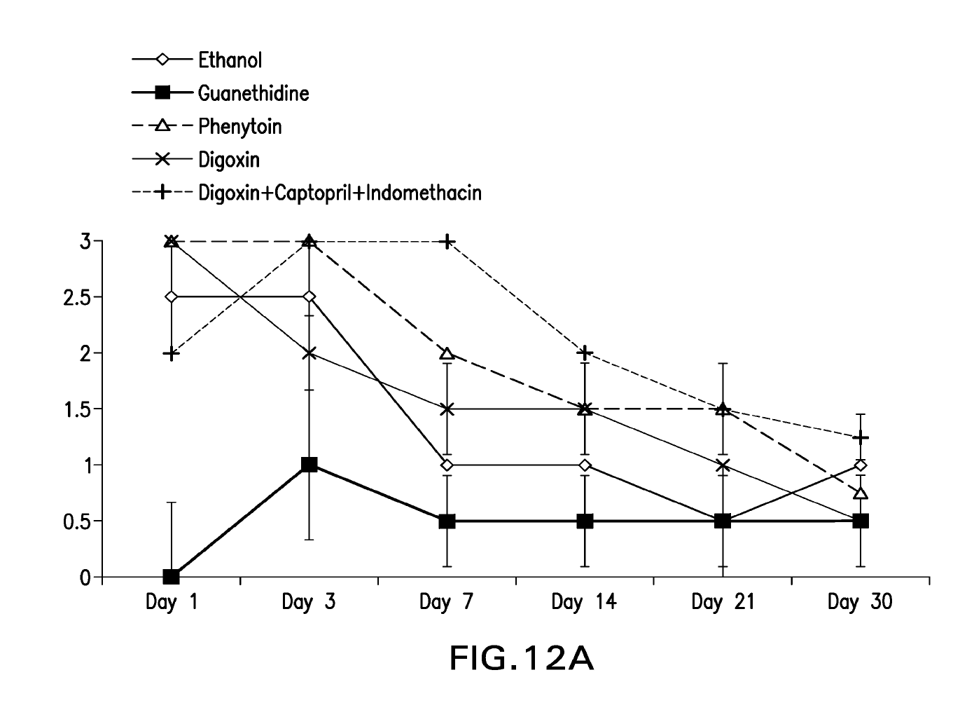

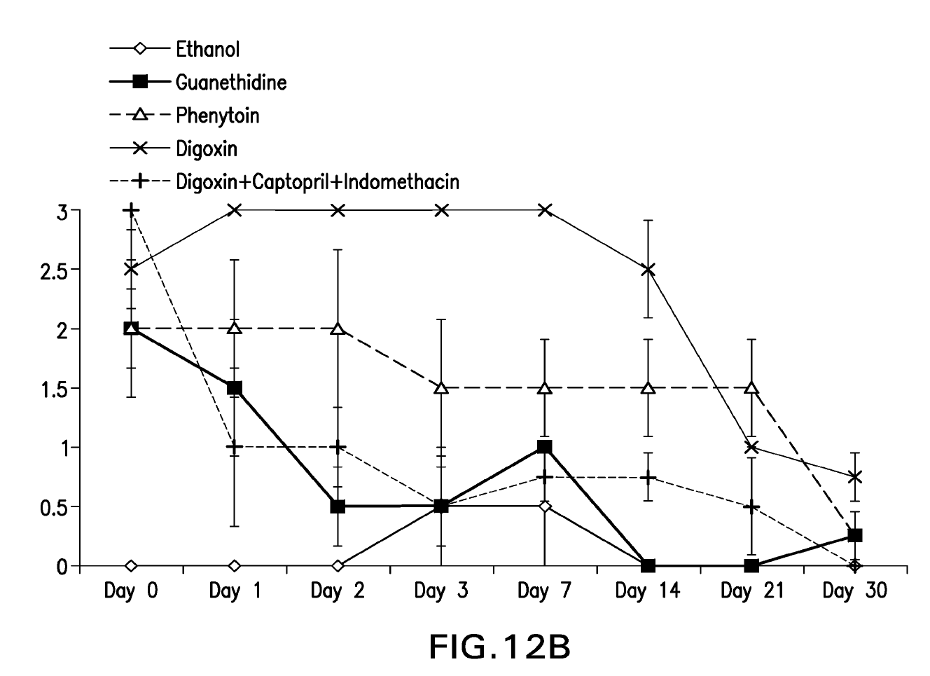

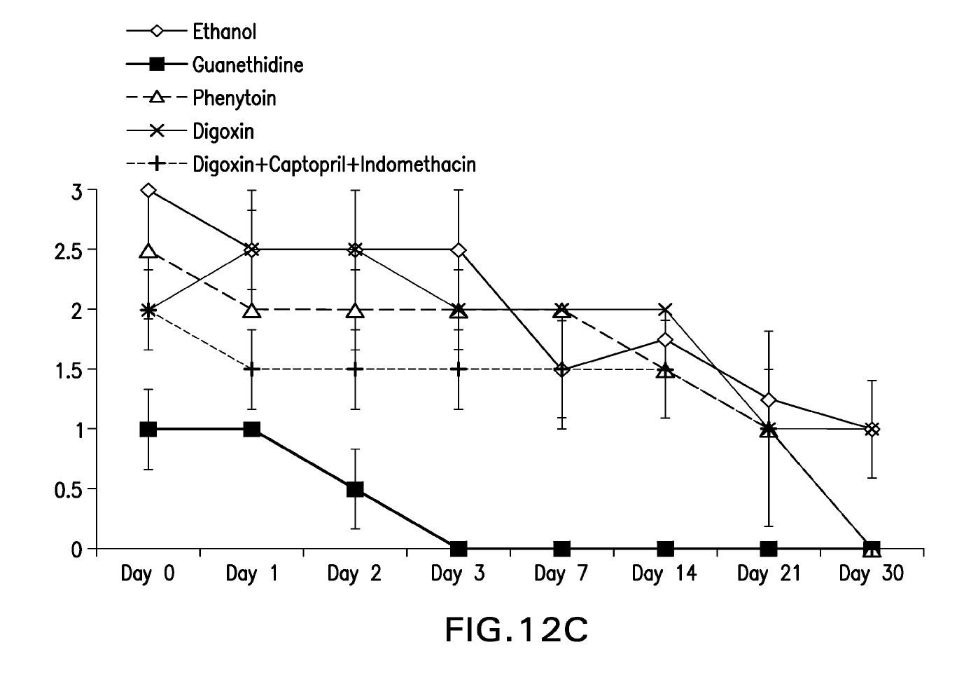

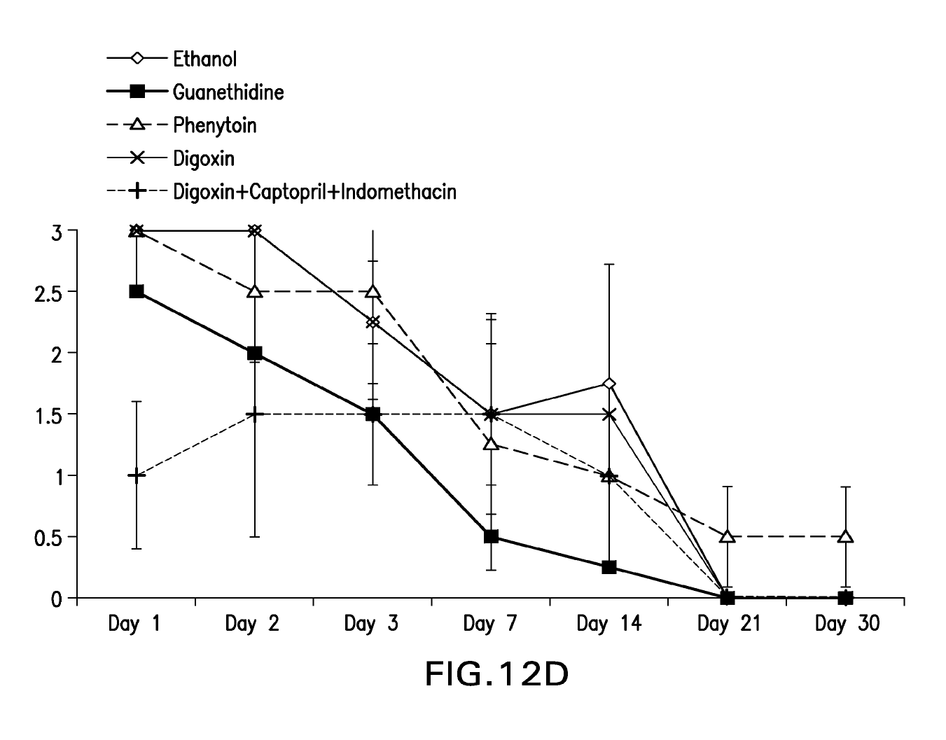

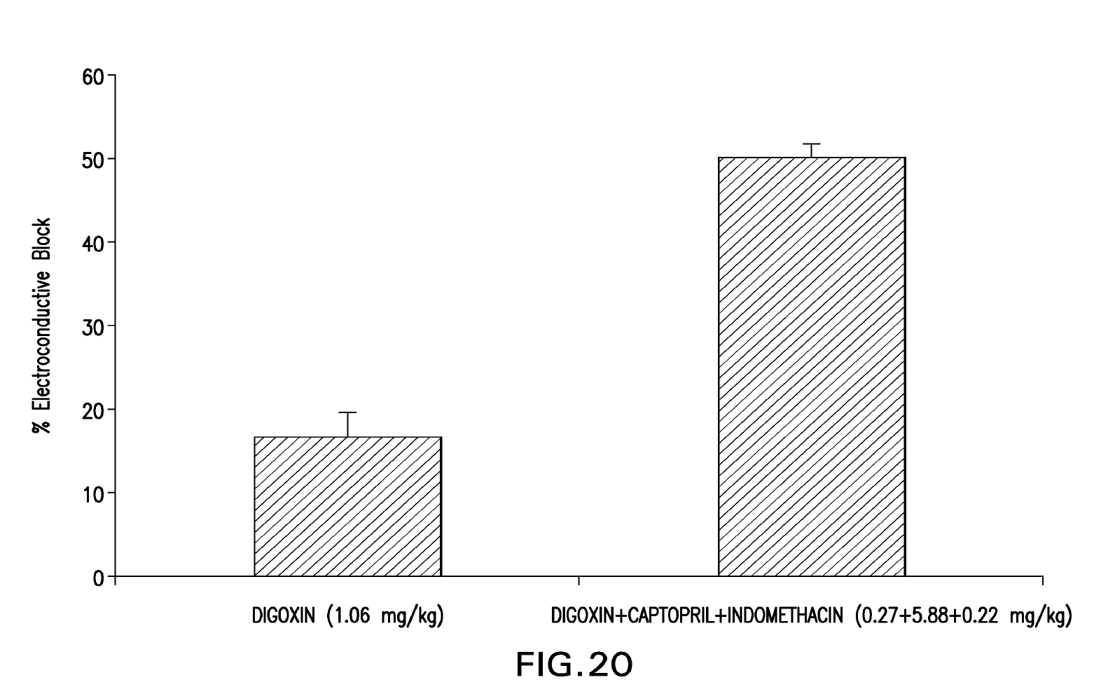

As previously stated, compositions for affecting nerve function may include a single agent, as well as a combination of two or more agents. There may be several advantages to the use of combinatorial agents to affect the function of nerve cells. First, different agents may act on different targets on the nerve cells and improve the efficacy of action. Second, there may be synergistic effects in which a first agent prevents firing (release of neurotransmitters, polarization, and/or opening of channels) of the nerve cells and a second agent prevents repolarization. Third, the synergistic effect of two or more agents may unexpectedly allow the concentration of the agents within the formulation to be lowered compared to use of a single agent (at a higher dose), while still achieving a desired efficacy. For instance, as disclosed in Example 1 and FIG. 12A, digoxin at a concentration of 1 mg/kg combined with captopril and indomethacin was superior in blocking nerve conductance than digoxin alone at a concentration of 3 mg/kg. In addition, referring to FIG. 20, data is provided showing that a low dose combination of digoxin (1.06 mg/kg), captopril (5.88 mg/kg), and indomethacin (0.22 mg/kg) is more effective in blocking nerve conduction than digoxin alone at a higher concentration of 1.06 mg/kg (approximately 50% conductive block vs. approximately 18% conductive block). Example 1 and FIGS. 12A-12D further show the synergistic effect of the combination of digoxin, captopril, and indomethacin because the combination outperformed guanethidine in the ability to affect peripheral nerve function.

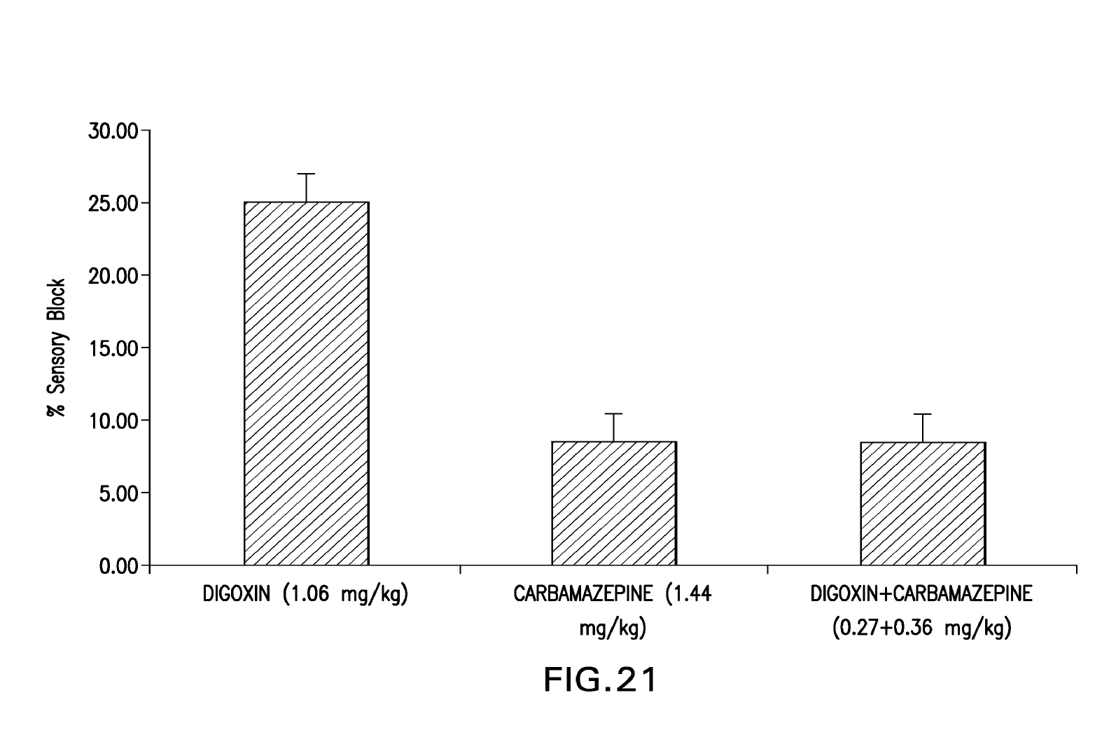

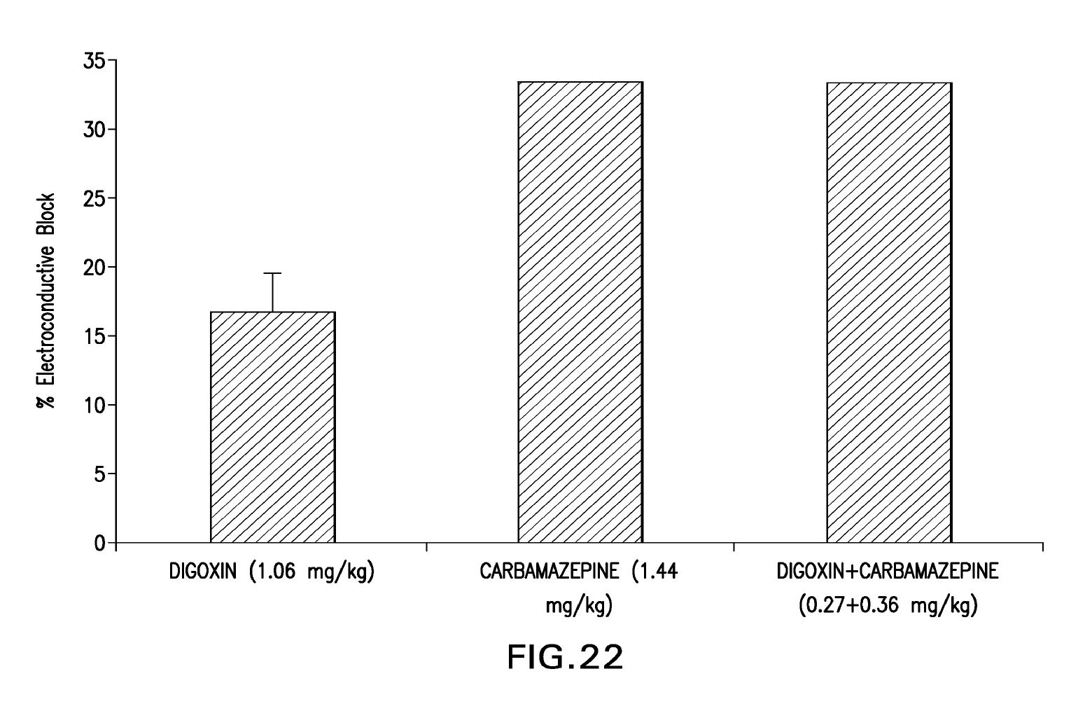

As mentioned above, it was surprising to find that a composition including a combination of digoxin and other agents could affect nerve function since digoxin has not been previously known to affect nerve (e.g., peripheral nerve and renal nerve) function. The agent combination of digoxin (cardiac glycoside), captopril (ACE-inhibitor), and indomethacin (NSAID) may be particularly beneficial in blocking or at least partially blocking nerve (e.g., renal nerve) function. However, not all agent combinations act synergistically or can be predicted to act synergistically. For example, as shown in FIG. 21, the amount of sensory block affected with a combination of digoxin (cardiac glycoside) and carbamazepine (sodium channel blocker) was not greater then when each agent was used alone. Furthermore, not all low dose combinations of agents are more effective than a high dose of the agents used alone. As shown in FIG. 22, the degree of electroconductive block when a low dose combination of digoxin (0.27 mg/kg) and carbamazepine (0.36 mg/kg) was administered was greater than digoxin alone at a higher dose (1.06 mg/kg) but not carbamazepine alone at a higher dose (1.44 mg/kg). Furthermore, agents in the same class may have varying or different effects on nerve function when administered alone or in combination. For instance, substituting ouabain for digoxin (cardiac glycosides) in compositions including agent combinations may not result in the same nerve effect. Similarly, substituting verapamil for diltiazem (calcium channel blockers) in multi-agent compositions may not result in the same nerve effect. In a similar manner, substituting colistin for metronidazole (antibiotics) in multi-agent compositions may not result in the same nerve effect and furthermore, substituting ciprofloxicin for moxifloxacin (both members of the quinolone class of antibiotics) in multi-agent compositions may have non-similar effects on nerves.

Durability of the effect of the nerve affecting compositions may be achieved by the administration of blocking agents in combination or in sequence with a durability agent. For example, one or a combination of blocking agents may be administered simultaneously with a durability agent. In another example, one or a combination of blocking agents can be administered and one or a combination of durability agents can be administered after a period of time. Sequential administration of durability agents may be achieved through local or systemic routes of administration at desirable concentrations. Durability agents may also be administered at various timepoints that may or may not coincide with the inflammatory response initiated by impaired nerve function and axonal degeneration.

Durability can also be achieved by the administration of blocking agents at a concentration that does not cause nerve cell lysis. Methods involving energy (i.e., ultrasound, RF, etc.) can cause nerve cell lysis and trigger local inflammation, which can increase nerve cell re-growth. Methods involving nerve cell block that do not involve cytolysis can be more durable.

When a combination of nerve affecting agents are employed, the ratio of agents in the compositions may be as follows.

| | | Percent |
|---|---|---|
| Combo | Agent | of total |
| 1 | Digoxin | 75% |
| | Captopril | 25% |
| 2 | Digoxin | 50% |
| | Phenytoin | 25% |
| | Captopril | 25% |
| 3 | Digoxin | 30% |
| | Chloroquin | 30% |
| | Verapamil | 20% |
| | Lithium Chloride | 20% |
| 4 | Digoxin | 30% |
| | Phenytoin | 20% |
| | Chloroquin | 20% |
| | Indomethacin | 10% |
| | Labetalol | 20% |
| 5 | Digoxin | 46% |
| | Captopril | 28% |
| | Indomethacin | 26% |

Other suitable ratios of nerve affecting agents may also be used in the compositions. With respect to dosing, the agents may be dosed at the FDA-approved loading dose. In other variations, the agents may be dosed at the FDA-approved intravenous dose. In yet further variations, agents may be dosed at the FDA-approved oral dose. For example, the patient may be dosed with 0.6 mg digoxin. Doses can be administered in multiple parts or to multiple locations. For example, 0.4 mg digoxin can be delivered into the wall of one renal artery, and 0.2 mg digoxin can be dosed into the wall of the other renal artery. In other variations, a mixture of agents can be made and the composition administered in equal or unequal parts. In yet further variations, agents or mixtures of agents can be made, dosed and administered by multiple routes. For example, one agent and dose may be administered parenterally (intra-arterial route using a catheter) and another agent and dose administered orally or combinations thereof.

In some variations, the nerve affecting compositions include a single agent, e.g., a cardiac glycoside. In other variations, the nerve affecting compositions include a combination of at least two agents or at least three agents. Nerve affecting compositions including more than three agents may also be used. Any suitable combination of agents may be employed. For example, use of the combination of a cardiac glycoside and an ACE-inhibitor may be beneficial. In further variations, a cardiac glycoside may be combined with one or more of nerve affecting agents selected from the group consisting of calcium channel blockers, sodium channel blockers, potassium channel blockers, angiotensin-converting enzyme (ACE) inhibitors, antibiotics, excitatory amino acids, and nonsteroidal anti-inflammatory drugs (NSAIDS), alpha-adrenergic blockers, beta-adrenergic blockers, benzodiazepines, nitroglycerin, amyl nitrate, pentaerythritol tetranitrate, and magnesium sulfate.

In one variation, the composition for affecting nerve function includes: (1) digoxin (a cardiac glycoside), (2) captopril (an ACE inhibitor), and (3) indomethacin (an NSAID). The digoxin dose may be approximately 0.2-2.0 mg/kg. The captopril dose may be approximately 2-20 mg/kg. The indomethacin dose may be approximately 0.2-20 mg/kg.

In another variation, the composition for affecting nerve function includes: (1) digoxin (a cardiac glycoside), and (2) indomethacin (an NSAID).

In yet a further variation, the composition for affecting nerve function includes: (1) digoxin (a cardiac glycoside), and (2) lithium chloride (a sodium channel blocker).

Other variations of the compositions for affecting nerve function include: (1) ouabain (a cardiac glycoside), (2) carbamazepine (a sodium channel blocker), and (3) captopril (an ACE inhibitor).

Some variations of the compositions for affecting nerve function include: (1) metronidazole (an antibiotic), (2) captopril (an ACE inhibitor), and (3) indomethacin (an NSAID).

In another variation, the composition for affecting nerve function includes: (1) digoxin (a cardiac glycoside), (2) lithium chloride (a sodium channel blocker), and (3) amlodipine (a calcium channel blocker).

Digoxin may be combined with various agents (one or more), including but not limited to: antibiotics such as aminoglycosides, amphenicols, ansamycins, lactams, lincosamides, macrolides, nitrofurans, quinolones, sulfonamides, sulfones, tetracyclines, and any of their derivatives; antifungal agents such as allylamines, imidazoles, polyenes, thiocarbamates, triazoles, and any of their derivatives; steroidal agents such as prednisone, methylprednisolone, solumedrol, triamcinolone, betamethasone, and the like; cytokines such as interferon alpha-2a, interferon alpha-2b, interferon beta-1a, interferon beta-1b, interferon gamma, and the like; antibodies such as rituximab, adalimumab, infliximab, alefacept, etanercept, and the like; gamma globulin; statins such as atorvastin, fluvastatin, lovastatin, mevastatin, pravastatin, rosuvastatin, simvastatin, and the like; fenofibrate; gemfibrozil; niacin; niacinamide; nicotine; antihistamines such as diphenhydramine, triprolidine, tripelenamine, fexofenadine, chlorpheniramine, doxylamine, cyproheptadine, meclizine, promethazine, phenyltoloxamine, hydroxyzine, brompheniramine, dimenhydrinate, cetirizine, loratadine, and the like; antidiabetes agents such as acarbose, glimepride, glyburide, metformin, miglitol, pioglitazone, repaglinide, rosiglitazone, and the like; nonsteroidal anti-inflammatory agents such as aspirin, salicylic acid, salsalate, diflunisal, ibuprofen, indomethacin, oxaprozin, sulindac, ketorolac, ketoprofen, nabumetone, piroxicam, naproxen, diclofenac, celecoxib, rofecoxib, valdecoxib, and the like; immunomodulatory agents such as cyclosporine, tacrolimus, pimecrolimus, levamisole, mycophenolate mofetil, methotrexate, cyclophosphamide, azathioprine, hydroxychloroquine, aurothioglucose, auranofin, penicillamine, sulfasalazine, leflunomide, sirolimus, paclitaxel, docetaxel, and the like; beta adrenergic inhibitors such as atenolol, betaxolol, bisoprolol, carvedilol, esmolol, labetalol, metoprolol, nadolol, pindolol, propanolol, sotalol, timolol, and the like; cholinergics such as bethanechol, oxotremorine, methacholine, cevimeline, carbachol, galantamine, arecoline, and the like; muscarine; pilocarpine; anticholinesterases such as edrophonium, neostigmine, donepezil, tacrine, echothiophate, demecarium, diisopropylfluorophosphate, pralidoxime, galanthamine, tetraethyl pyrophosphate, parathion, malathion, isofluorophate, metrifonate, physostigmine, rivastigmine, abenonium acetylchol, carbaryl acetylchol, propoxur acetylchol, aldicarb acetylchol, and the like; calcium channel blockers such as amlodipine, diltiazem, felodiipine, isradipine, nicardipine, nifedipine, nisoldipine, verapamil, and the like; sodium channel blockers such as moricizine, propafenone, encainide, flecainine, tocainide, mexilietine, phenytoin, lidocaine, disopyramine, quinidine, procainamide, and the like; mifepristone; vesicular monoamine transport agents such as guanadrel, guanethidine, reserpine, mecamylamine, hexemethonium, and the like; hydralazine; minoxidil; combination adrenergic inhibitors such as labetalol, carvedilol, and the like; alpha-adrenergic blockers such as doxazosin, prazosin, terazosin, and the like; nitrate derivatives such as L-arginine; nitroglycerine, isosorbide, mononitrate, dinitrate, tetranitrate, and the like; endothelin receptor antagonists such as ambrisentan, bosentan, and the like; phosphodiesterase inhibitors such as vardenafil, tadalafil, sildenafil, and the like; spironolactone, eplerenone, and the like; angiotensin receptor antagonists such as candesartan, irbesartan, losartan, telmisartin, valsartan, eprosartan, and the like; ACE inhibitors such as benazepril, captopril, enalapril, fosinopril, lisinopril, moexipril, quinapril, ramipril, trandolapril, and the like; neurotoxins such as resinoferatoxin, alpha-bungarotoxin, tetrodotoxin, botulinum toxin, and the like; renin inhibitors such as aliskiren, and the like; anticoagulants such as heparin, low molecular weight heparin, fondaparinux, coumadin, acenocoumarol, phenprocoumon, phenindione, argatroban, lepirudin, bivalirudin, clopidogrel, ticlopidine, cilostazol, abciximab, eptifibatide, tirofiban, dipyridamole, and the like; thrombolytic agents such as alteplase, reteplase, urokinase, streptokinase, tenectaplase, lanoteplase, anistreplase, and the like; leukotriene antagonists such as montelukast, zafirlukast, and the like; agents that influence the autonomic nervous system such as beta-blockers, aldosterone antagonists, angiotensin II receptor blockades, angiotensin converting enzyme (“ACE”) inhibitors, endothelin receptor antagonists, sympathomimetics, calcium channel blockers; sodium channel blockers, vasopressin inhibitors, peripheral adrenergic inhibitors; oxytocin inhibitors, botulinum toxin, statins, triglyceride lowering agents, niacin, diabetes agents, immunomodulators, nicotine, sympathomimetics, antihistamines, cholinergics, acetylcholinesterase inhibitors, magnesium and magnesium sulfates, calcium channel blockers, muscarinics, sodium channel blockers, glucocorticoid receptor blockers, blood vessel dilators, central agonists, combined alpha and beta-blockers, alpha blockers, combination diuretics, potassium sparing diuretics, cyclic nucleotide monophosphodiesterase inhibitors, alcohols, vasopressin inhibitors, oxytocin inhibitors, glucagon-like peptide 1, relaxin, renin inhibitors, estrogen and estrogen analogues and metabolites, progesterone inhibitors, testosterone inhibitors, gonadotropin-releasing hormone analogues, gonadotropin-releasing hormone inhibitors, type 4 phosphodiesterase inhibitors, vesicular monoamine transport inhibitors, melatonin, anticoagulants, beta agonists, alpha agonists; indirect agents that include norepinephrine, epinephrine, norepinephrine, acetylcholine, sodium, calcium, angiotensin I, angiotensin II, angiotensin converting enzyme I, angiotensin converting enzyme II, aldosterone, potassium channel blockers and magnesium channel blockers, cocaine, amphetamines, ephedrine, terbutaline, dopamine, dobutamine, antidiuretic hormone, oxytocin, and THC cannabinoids.

Additional agents that may be combined with digoxin include beta-blockers: atenolol (e.g., as sold under the brand names Tenormin), betaxolol (e.g., as sold under the brand name Kerlone), bisoprolol (e.g., as sold under the brand name Zebeta), carvedilol (e.g., as sold under the brand name Coreg), esmolol (e.g., as sold under the brand name Brevibloc), labetalol (e.g., as sold under the brand name Normodyne), metoprolol (e.g., as sold under the brand name Lopressor), nadolol (e.g., as sold under the brand name Corgard), pindolol (e.g., as sold under the brand name Visken), propranolol (e.g., as sold under the brand name Inderal), sotalol (e.g., as sold under the brand name Betapace), timolol (e.g., as sold under the brand name Blocadren), carvedilol, and the like; aldosterone antagonists: e.g., spironolactone, eplerenone, and the like; angiotensin II receptor blockades: e.g., candeartan (e.g., available under the brand name Altacand), eprosarten mesylate (e.g., available under the brand name Tevetan), irbesartan (e.g., available under the brand name Avapro), losartan (e.g., available under the brand name Cozaar), etelmisartin (e.g., available under the brand name Micardis), valsartan (e.g., available under the brand name Diovan), and the like; angiotensin converting enzyme (“ACE”) inhibitors: e.g., benazapril (e.g., available under the brand name Lotensin), captopril (e.g., available under the brand name Capoten), enalapril (e.g., available under the brand name Vasotec), fosinopril (e.g., available under the brand name Monopril), lisinopril (e.g., available under the brand name Prinivil), moexipril (e.g., available under the brand name Univasc), quinapril (e.g., available under the brand name AccupriL), ramipril (e.g., available under the brand name Altace), trandolapril (e.g., available under the brand name Mavik), and the like; sympathomimetics: e.g., trimethaphan, clondine, reserpine, guanethidine, and the like; calcium channel blockers: e.g., amlodipine besylate (e.g., available under the brand name Norvasc), diltiazem hydrochloride (e.g., available under the brand names Cardizem CD, Cardizem SR, Dilacor XR, Tiazac), felodipine plendil isradipine (e.g., available under the brand names DynaCirc, DynaCirc CR), nicardipine (e.g., available under the brand name Cardene SR), nifedipine (e.g., available under the brand names Adalat CC, Procardia XL), nisoldipine sulfur (e.g., available under the brand name Sular), verapamil hydrochloride (e.g., available under the brand names Calan SR, Covera HS, Isoptin SR, Verelan) and the like; sodium channel blockers: e.g., moricizine, propafenone, encainide, flecainide, tocainide, mexiletine, phenytoin, lidocaine, disopyramide, quinidine, procainamide, and the like; vasopressin inhibitors: e.g., atosiban (Tractocile), AVP V1a (OPC-21268, SR49059 (Relcovaptan)), V2 (OPC31260, OPC-41061 (Tolvaptan), VPA-985 (Lixivaptan), SR121463, VP-343, FR161282) and mixed VlaN2 (YM-087 (Conivaptan), JTV-605, CL-385004) receptor antagonists, and the like; peripheral adrenergic inhibitors: e.g., guanadrel (e.g., available under the brand name Hylorel), guanethidine monosulfate (e.g., available under the brand name Ismelin), reserpine (e.g., available under the brand names Serpasil, Mecamylamine, Hexemethonium), and the like; blood vessel dilators: e.g., hydralazine hydrocholoride (e.g., available under the brand name Apresoline), minoxidil (e.g., e.g., available under the brand name Loniten), and the like; central agonists: e.g., alpha methyldopa (e.g., available under the brand name Aldomet), clonidine hydrochloride (e.g., available under the brand name Catapres), guanabenz acetate (e.g., available under the brand name Wytensin), guanfacine hydrochloride (e.g., available under the brand name Tenex), and the like; combined alpha and beta-blockers: e.g., carvedilol (e.g., available under the brand name Coreg), labetolol hydrochloride (e.g., available under the brand names Normodyne, Trandate), and the like; alpha blockers: e.g., doxazosin mesylate (e.g., available under the brand name Cardura), prazosin hydrochloride (e.g., available under the brand name Minipress), terazosin hydrochloride (e.g., available under the brand name Hytrin), and the like; renin inhibitors: e.g., Aliskiren, and the like; oxytocin inhibitors: e.g., terbutaline, ritodrine, and the like, and botulism toxin (or botox) and the like.

Other potential agents that may be delivered in combination with digoxin are smooth muscle relaxants that may include, but are not limited to, alvarine, anisotropine, atropine, belladonna, clidinium, dicyclomine, glycopyrrolate, homatropine, hyoscyamine, mebevarine, mepenzolate, methantheline, methscopolamine, oxybutynin, papavarine, pirenzepine, popantheline, scopolamine, and the like.

Furthermore, digoxin may be combined with any number of chemotherapeutic agents, specifically those cytotoxic agents traditionally used to treat cancer. Such agents may include, but are not limited to, alkylating agents such as busulfan, hexamethylmelamine, thiotepa, cyclophosphamide, mechlorethamine, uramustine, melphalan, chlorambucil, carmustine, streptozocin, dacarbazine, temozolomide, ifosfamide, and the like; anti-metabolites such as methotrexate, azathioprine, mercaptopurine, fludarabine, 5-fluorouracial, and the like; anthracyclines such as daunorubicin, doxorubicin, epirubicin, idarubicin, mitoxantrone, and the like; plant alkaloids and terpenoids such as vincristine, vinblastine, vinorelbine, vindesine, podophyllotoxin, paclitaxel, doclitaxel, and the like; topoisomerase inhibitors such as irinotecan, amsacrine, topotecan, etoposide, teniposide, and the like; antibody agents, such as rituximab, trastuzumab, bevacizumab, erlotinib, dactinomycin; finasteride; aromatase inhibitors; tamoxifen; goserelin; and imatinib mesylate.

### IV. ADDITIVES

For local delivery performed under fluoroscopy, small amounts of radiopaque contrast agents (commercially available agents like Omnipaque and others) may be included in the nerve affecting compositions described herein without compromising their efficacy. These contrast agents provide visual confirmation that the agent is being delivered to the target location during the clinical procedure. Both ionic and non-ionic contrast agents can be used. Examples include diatrizoate (Hypaque 50), metrizoate (Isopaque 370), ioxaglate (Hexabrix), iopamidol (Isovue 370), iohexol (Omnipaque 350), ioxilan (Oxilan 350), iopromide (Ultravist 370), and iodixanol (Visipaque 320).

In some instances, a carrier may be included in the nerve affecting compositions. Exemplary carriers include without limitation, dimethyl sulfoxide (0-99% v/v), ethanol (0-99% v/v), acetone (0-10% v/v), normal saline (0-90% w/v), water (0-50% v/v), methylcellulose (0-30% w/v), albumin (0-20% w/v), and deoxycholate (0-10% w/v).

In some instances, a carrier may include a small amount of anesthetic agent to immediately block the sensory signals and prevent any possible pain experienced by the patient during the procedure. Renal denervation using RF ablation is very painful to the patient and is performed under sedation using an anesthetic. Exemplary anesthetic agents include without limitation, lidocaine, prilocaine, bupivacaine and ropivacaine.

The nerve affecting compositions take any suitable form. For example, the nerve affecting compositions may be made as a solution, suspension, emulsion, microspheres, liposomes, etc. The nerve affecting compositions may also be formulated for any suitable type of release, e.g., sustained release, controlled release, or delayed release of the nerve affecting agent(s). In other variations, microbubbles may be included in the agent compositions to enhance their visualization at the target site.

In some variations, the compositions including one or more nerve affecting agents are time-release formulations formed as microspheres. The microspheres are made from biodegradable polymer matrices containing the agents, bioerodible matrices, and biodegradable hydrogels or fluids that have prolonged agent release rates and degradation profiles. The agent is released as the polymer degrades and non-toxic residues are removed from the body over a period of week to months. Useful polymers for the biodegradable controlled release microspheres for the prolonged administration of agents to a targeted site include polyanhydrides, polylactic acid-glycolic acid copolymers, and polyorthoesters. Polylactic acid, polyglycolic acid, and copolymers of lactic acid and glycolic acid are preferred. Other polymer matrices include polyethylene glycol hydrogels, chitin, and polycaprolactone copolymers.

### V. DEVICES

The devices used to deliver the nerve affecting compositions described herein may be generally configured for percutaneous advancement through the vasculature. The devices may include an expandable element having an expanded configuration and an unexpanded configuration. The expandable element may be self-expanding or expanded by infusion of a fluid, e.g., saline or a contrast solution, or by mechanical actuation. Mechanical actuation may be effected by slideable caps coupled to the proximal and distal ends of the expandable element, as further described below. In some variations, the expandable element is a balloon. In other variations, the expandable element is a radially expandable cage or frame. The delivery devices may be configured to include one or more expandable elements. Thus in further variations, the expandable element includes both a balloon and a radially expandable cage or frame. The cage or frame may comprise one wire or a plurality of wires that twist, turn, or helically spiral around the exterior wall of the balloon, and which radially expand upon inflation of the balloon. The wires may be solid or hollow. The expandable element as well as other components of the delivery devices may be preformed to have any suitable geometry and dimensions.

The expandable elements may include one or more needle housings for containing a slideable needle. The needle housings may be located in the wall of the expandable element or provided on its surface. In some variations, the radially expandable cage or frame is the slidable needle housing. The needle housings may be configured in any suitable manner on the delivery devices. For example, in some variations, the needle housings are placed on the surface of the expandable element in a helical or spiral fashion. Furthermore, the slideable needles may take any suitable form, e.g., straight, curved, angled at 90 degrees, preformed, etc., and may exit the needle housings at any location along the axial length of the housing. In some variations, the slideable needles exit the needle housings so that injection into the vascular wall occurs in a helical or spiral pattern.

The delivery devices may be configured to deliver a nerve affecting composition including a single agent or a plurality of agents. For example, the delivery devices may deliver a nerve affecting composition comprising a cardiac glycoside, a calcium channel blocker, a NSAID. Here the cardiac glycoside may be digoxin, the calcium channel blocker may be captopril, the NSAID may be indomethacin. In this and other variations, the nerve affecting agents may be combined to form a single composition and then injected using the devices described herein. The nerve affecting agents may also be delivered (injected) separately using the same or different devices. The nerve affecting agents may further be delivered to the target tissue simultaneously or sequentially. Sequential administration of agents may be done immediately without delay or delayed by a specified time period varying between a few days to 2 months using local or systemic routes for delivery.

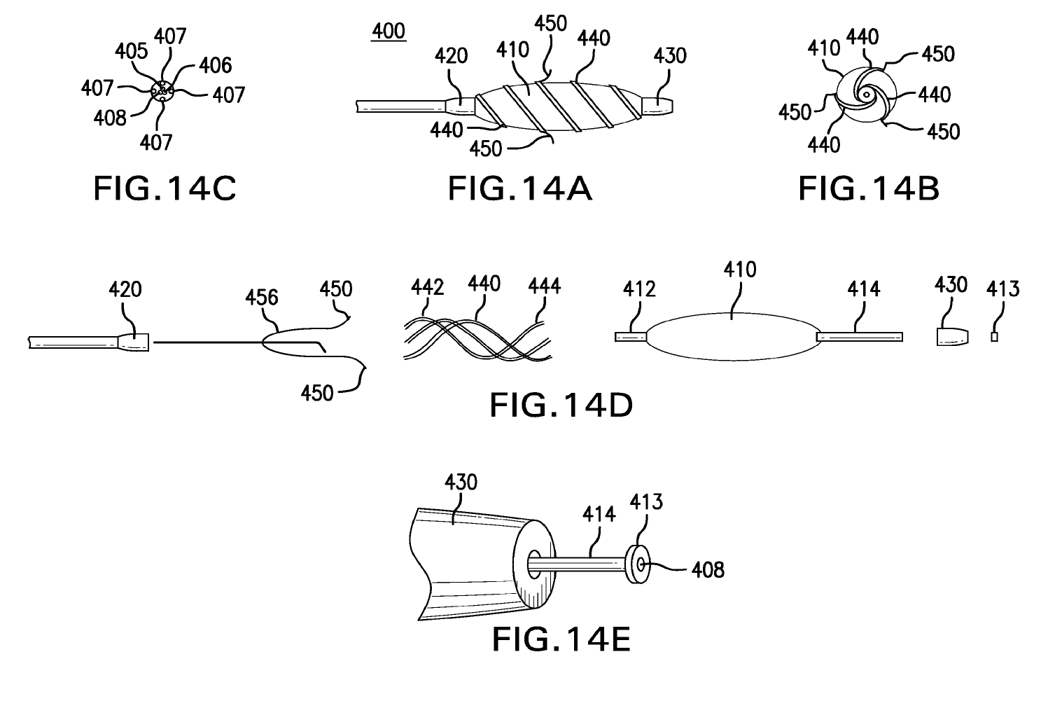

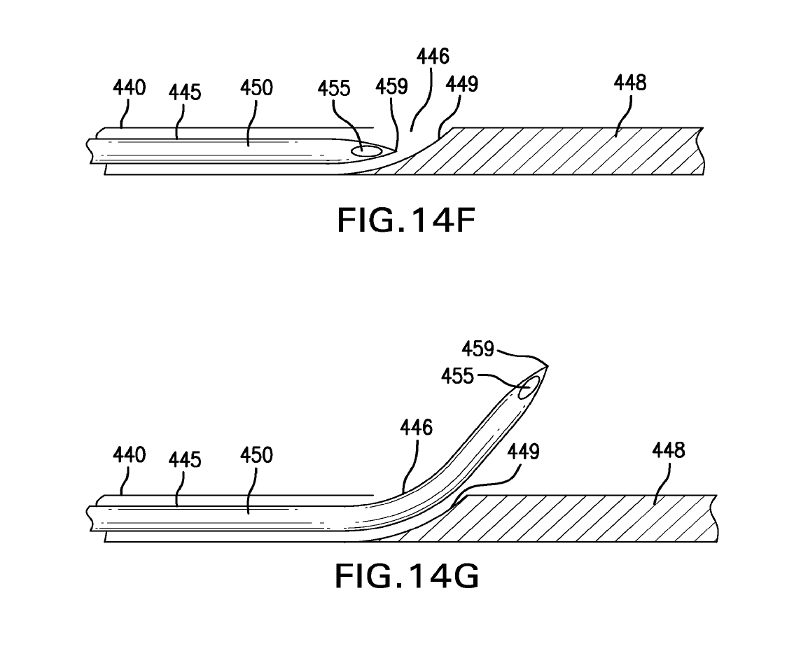

One variation of a delivery device, comprising delivery catheter 400, is shown in FIGS. 14A-14H. FIGS. 14A-14B show side and end views of delivery catheter 400. Delivery catheter 400 includes a balloon 410, a proximal cap 420, a distal cap 430, a plurality of needle housings 440, and a plurality of delivery needles 450.

FIG. 14C shows another end view of delivery catheter 400. Delivery catheter 400 includes a needle lumen 405 and an inflation lumen 406. Delivery catheter may also include one or more steering lumens 407 and a guidewire lumen 408.

FIG. 14D shows an assembly view of delivery catheter 400. Balloon 410 includes a proximal portion 412 and a distal portion 414. Proximal cap 420 is coupled to proximal portion 412 of balloon 410. Distal cap 430 is slidably coupled to distal portion 414 of balloon 410. Distal portion 414 of balloon 410 may include a stop 413 which prevents distal cap 430 from sliding off. Needle housings 440 have a substantially helical configuration and are disposed about the outer wall of the balloon 410. Each needle housing 440 includes a proximal portion 442 and a distal portion 444. Proximal portions 442 of needle housings 440 are coupled to proximal cap 420. Distal portions 444 of needle housings 440 are coupled to distal cap 430. Each needle housing 440 includes a needle lumen 445. A delivery needle 450 is slidably disposed within each needle lumen 445. Delivery needles 450 may be coupled to a manifold 456 which distributes an agent to delivery needles 450.

FIG. 14E shows an enlarged view of distal cap 430. Distal cap 430 freely slides along and rotates around distal portion 414 of balloon 410.

FIGS. 14F-14G show enlarged views of needle housing 440. Needle housing 440 includes a needle lumen 445 formed proximally to a needle port 446. Needle lumen 445 is in communication with needle port 446. Needle port 446 is formed in an outwardly-facing surface of needle housing 440. Delivery needle 450 may be advanced and withdrawn through needle port 446. Needle lumen 445 may include a ramp 449 which directs delivery needle 450 out through needle port 446. Needle housing 440 may include an imaging marker 448. Imaging marker 448 may be a radiopaque material, coating, or other suitable marker for aiding visualization of needle housing 440. Delivery needle 450 includes a delivery lumen 455. Delivery needle 450 includes a tip 459 configured to penetrate the wall of a vessel. FIG. 14F shows needle housing 440 with delivery needle 450 retracted. FIG. 14G shows needle housing 440 with delivery needle 450 advanced through needle port 446.

Balloon 410 is sufficiently rigid to maintain the spacing between proximal cap 420 and distal cap 430, yet flexible enough to bend 90 degrees or more. Like balloon 410, needle housings 440 are also flexible enough to bend 90 degrees or more, which allows delivery catheter 400 to navigate into branched vessels, such as from the aorta into the renal arteries. The position of the balloon 410 at the target site may be verified by infusing the balloon with a radiopaque fluid or contrast agent.

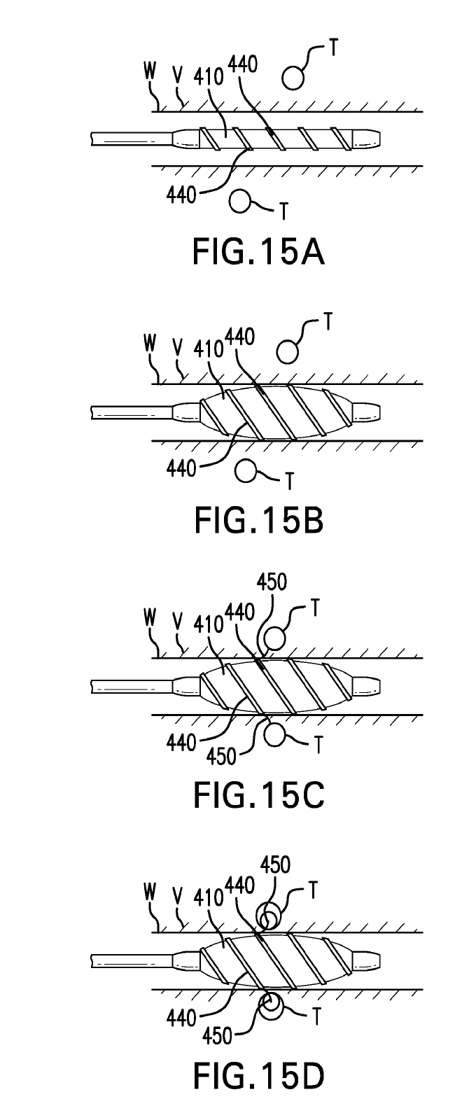

FIGS. 15A-15D show one variation of a method for using delivery catheter 400. FIG. 15A shows delivery catheter 400 advanced into a vessel V and balloon 410 positioned at or near one or more target sites T. FIG. 15B shows balloon 410 expanded and needle housings 440 brought into contact with walls W of vessel V. FIG. 15C shows delivery needles 450 advanced out of needle housings 440 and into the walls W. FIG. 15D shows delivery needles 450 delivering one or more agents to the target sites T. After delivery is complete, needles 450 are retracted back into needle housings 440 and balloon 410 deflated.

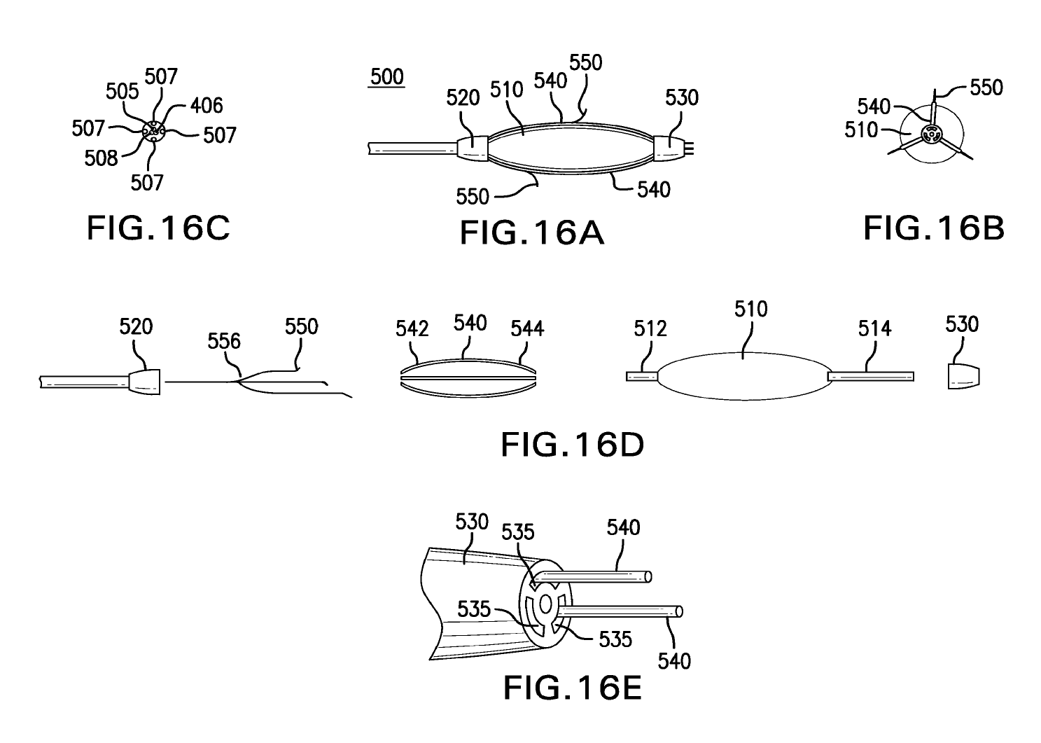

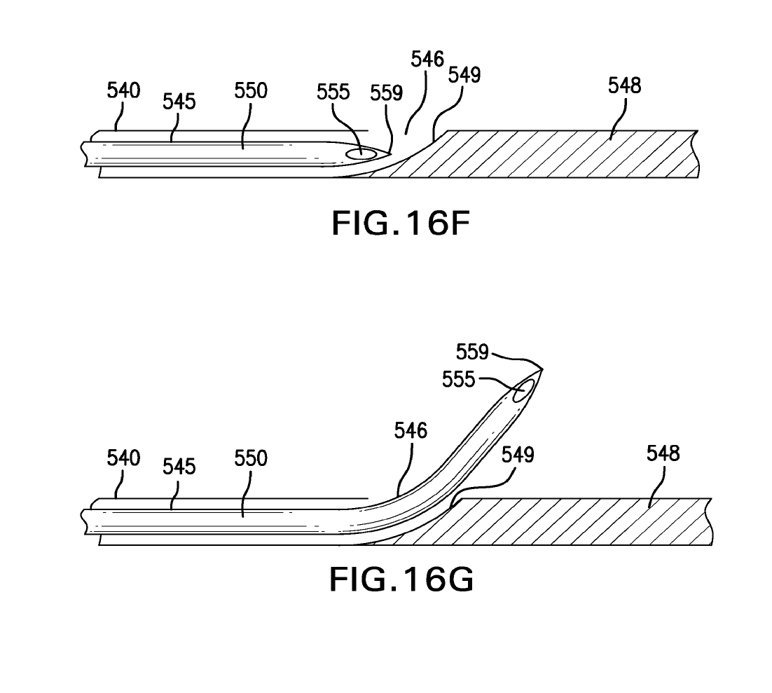

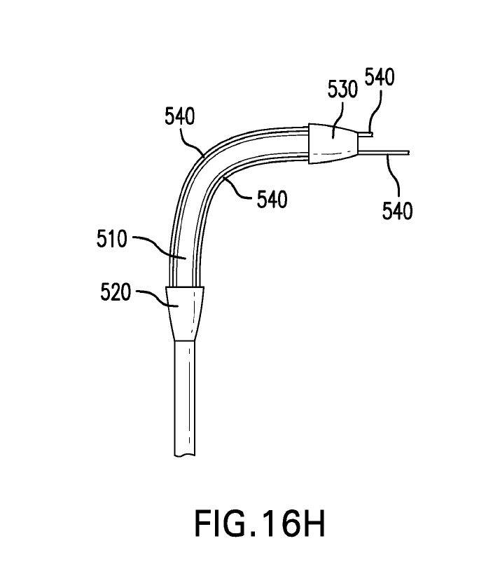

FIGS. 16A-16H show another variation of a delivery catheter 500.

FIGS. 16A-16B show side and end views of delivery catheter 500. Delivery catheter 500 includes a balloon 510, a proximal cap 520, a distal cap 530, a plurality of needle housings 540, and a plurality of delivery needles 550.

FIG. 16C shows another end view of delivery catheter 500. Delivery catheter 500 includes a needle lumen 505 and an inflation lumen 506. Delivery catheter may also include one or more steering lumens 507 and a guidewire lumen 508.

FIG. 16D shows an assembly view of delivery catheter 500. Balloon 510 includes a proximal portion 512 and a distal portion 514. Proximal cap 520 is coupled to proximal portion 512 of balloon 510. Distal cap 530 is coupled to distal portion 514 of balloon 510. Each needle housing 540 includes a proximal portion 542 and a distal portion 544. Proximal portions 542 of needle housings 540 are fixedly coupled to proximal cap 520. Distal portions 544 of needle housings 540 slide freely through distal cap 530. Each needle housing 540 includes a needle lumen 545. A delivery needle 550 is slidably disposed within each needle lumen 545. Delivery needles 550 may be coupled to a manifold 556 which distributes an agent to delivery needles 550.

FIG. 16E shows an enlarged view of distal cap 530. Distal cap 530 includes one or more openings 535 through which needle housings 540 may slide freely.

FIGS. 16F-16G show enlarged views of needle housing 540. Needle housing 540 includes a needle lumen 545 formed proximally to a needle port 546. Needle lumen 545 is in communication with needle port 546. Needle port 546 is formed in an outwardly-facing surface of needle housing 540. Delivery needle 550 may be advanced and withdrawn through needle port 546. Needle lumen 545 may include a ramp 549 which directs delivery needle 550 out through needle port 546. Needle housing 540 may include an imaging marker 548. Imaging marker 548 may be a radiopaque material, coating, or other suitable marker for aiding visualization of needle housing 540. Delivery needle 550 includes a delivery lumen 555. Delivery needle 550 includes a tip 559 configured to penetrate the wall of a vessel. FIG. 16F shows needle housing 540 with delivery needle 550 retracted. FIG. 16G shows needle housing 540 with delivery needle 550 advanced through needle port 546.

FIG. 16H shows delivery catheter 500 being bent at a 90 degree angle. Balloon 510 is sufficiently rigid to maintain the spacing between proximal cap 520 and distal cap 530, yet flexible enough to bend 90 degrees or more. Like balloon 510, needle housings 540 are also flexible enough to bend 90 degrees or more, which allows delivery catheter 500 to navigate into branched vessels, such as from the aorta into the renal arteries. Needle housings 540 slide freely through distal cap 530, which allows a needle housing 540 on the inside of a bend to slide further through distal cap 530, while allowing a needle housing 540 on the outside of a bend to slide not as far through distal cap 530. Distal cap 530 may be of sufficient length or otherwise configured to prevent distal portion 544 of needle housing 540 from sliding completely out of distal cap 530.

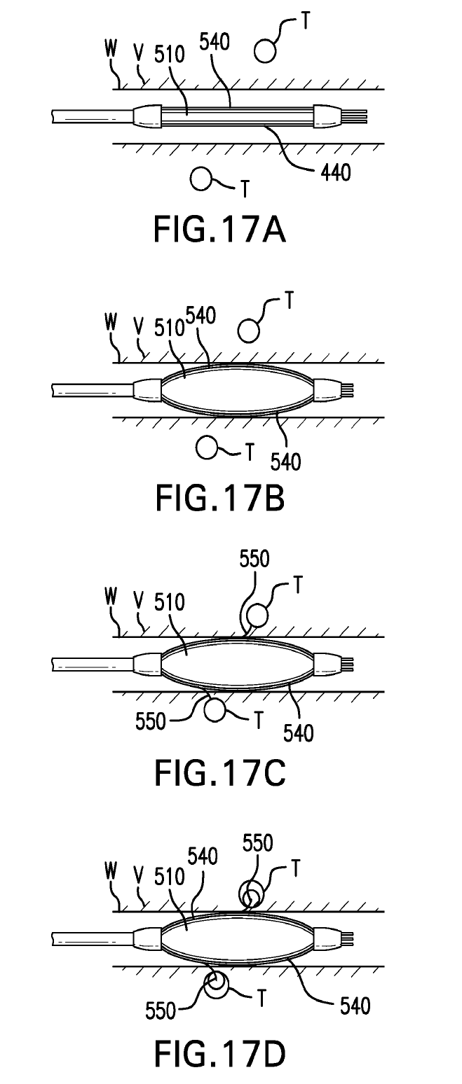

FIGS. 17A-17D show one variation of a method for using delivery catheter 500. FIG. 17A shows delivery catheter 500 advanced into a vessel V and balloon 510 positioned at or near one or more target sites T. FIG. 17B shows balloon 510 expanded and needle housings 540 brought into contact with walls W of vessel V. FIG. 17C shows delivery needles 550 advanced out of needle housings 540 and into the walls W. FIG. 17D shows delivery needles 550 delivering one or more agents to the target sites T. After delivery is complete, needles 550 are retracted back into needle housings 540 and balloon 510 deflated.

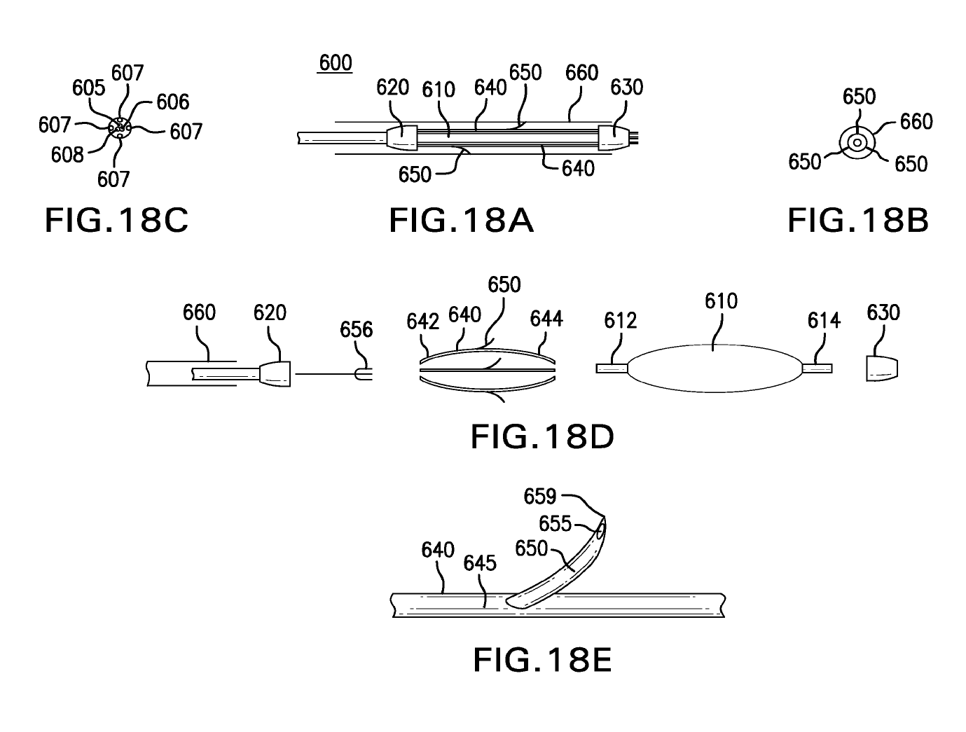

FIGS. 18A-18E show yet another variation of a delivery catheter 600.

FIGS. 18A-18B show side and end views of delivery catheter 600. Delivery catheter 600 includes a balloon 610, a proximal cap 620, a distal cap 630, a plurality of needle supports 640, a plurality of delivery needles 650, and a sheath 660.

FIG. 18C shows another end view of delivery catheter 600. Delivery catheter 600 includes a needle lumen 605 and an inflation lumen 606. Delivery catheter may also include one or more steering lumens 607 and a guidewire lumen 608.

FIG. 18D shows an assembly view of delivery catheter 600. Balloon 610 includes a proximal portion 612 and a distal portion 614. Proximal cap 620 is coupled to proximal portion 612 of balloon 610. Distal cap 630 is coupled to distal portion 614 of balloon 610. Each needle support 640 includes a proximal portion 642 and a distal portion 644. Proximal portions 642 of needle supports 640 are coupled to proximal cap 620. Distal portions 644 of needle supports 640 are coupled to distal cap 630. Each needle support 640 includes a delivery lumen 645. A delivery needle 650 is coupled to a side of each needle support 640 in fluid communication with delivery lumen 645. Delivery needles 650 are outwardly biased, and may be constrained or deployed by sheath 660 slidably positioned around delivery needles 650. Needle supports 640 may be coupled to a manifold 656 which distributes an agent to delivery lumens 645.

FIG. 18E shows an enlarged view of needle support 640 and delivery needle 650. Needle support 640 includes a delivery lumen 645 formed proximally to delivery needle 650. Delivery needle 650 includes a delivery lumen 655. Delivery lumen 645 of needle support 640 is in fluid communication with delivery lumen 655 of needle 650. Delivery needle 650 includes a tip 659 configured to penetrate the wall of a vessel. Needle support 640 may include an imaging marker 648. Imaging marker 648 may be a radiopaque material, coating, or other suitable marker for aiding visualization of needle support 640.

Balloon 610 is sufficiently rigid to maintain the spacing between proximal cap 620 and distal cap 630, yet flexible enough to bend 90 degrees or more. Like balloon 610, needle supports 640 are also flexible enough to bend 90 degrees or more, which allows delivery catheter 600 to navigate into branched vessels, such as from the aorta into the renal arteries.

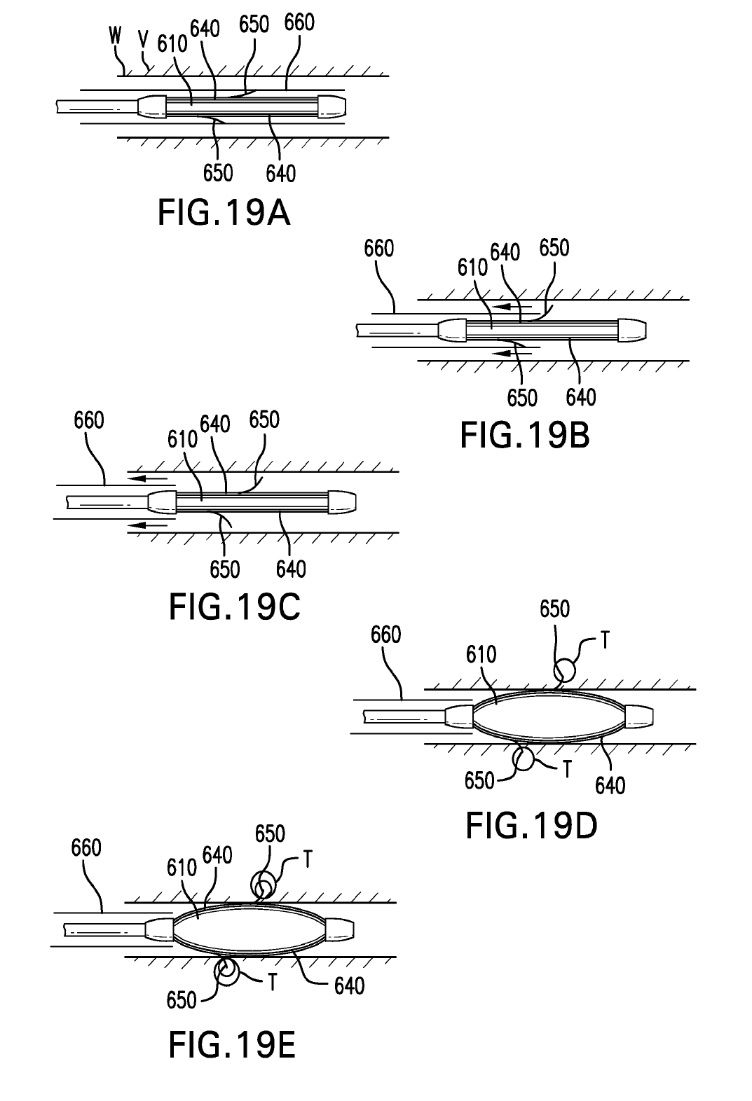

FIGS. 19A-19E show one variation of a method for using delivery catheter 600. FIG. 19A shows delivery catheter 600 advanced into a vessel V and balloon 610 positioned at or near one or more target sites T. The position of the balloon may be verified by inflating it with a radiopaque fluid or contrast agent. FIG. 19B shows sheath 660 partially retracted from delivery needles 650. FIG. 19C shows sheath 660 completely retracted from delivery needles 650, with delivery needles 650 pointing outwards. FIG. 19D shows balloon 610 expanded and delivery needles 650 forced into the walls W. FIG. 19E shows delivery needles 650 delivering one or more agents to the target sites T. After delivery is complete, balloon 610 is deflated and sheath 660 is advanced back over needles 650.

Delivery catheters 400, 500, and 600 are capable of injecting small volumes of agents, 0.005-0.5 ml, or 0.05-0.3 ml per injection site (or 0.05-3 ml total volume, or 0.5-1 ml total volume) to very localized sites within the body. These delivery catheters are capable of specifically targeting nerve cells and portions of the nerve cell, and locally affecting nerve function and provide therapeutic benefit from a degenerated and overactive sympathetic nervous system. Such low volumes reduce loss of agent into the systemic circulation and reduce damage to surrounding tissue and organs.

By contrast, tissue damage zones induced by radiofrequency ablation and guanethidine-induced denervation are quite macroscopic. RF ablation requires the creation of five to eight lesions along the renal artery; typical dimensions range between 2-3 mm in size. About 6 ml of guanethidine is injected into the vessel wall causing a large, single damage zone of about 10 mm. In addition, there may be significant pain associated with the RF ablation clinical procedure; patients are often sedated during ablation. The delivery catheters described above reduce tissue damage and pain during the procedure by precisely delivering microvolumes of agent per injection site without the need for sedation during a procedure.

Delivery catheters 400, 500, and 600 are: (i) sufficiently flexible to access the target site (the catheter is sufficiently flexible to access the renal arteries), (ii) small in profile, to minimize injury during introduction and delivery, (iii) configured to provide perfusion during agent delivery, (iv) constructed of materials which enhance visibility under fluoroscopy to help accurately position the device and deliver the agents to precise locations within the tissue, and (v) configured with needles of suitable quantity, locations, and depths for delivery and distribution of an agent to targeted sites (an anatomic location in a body, targeted sites within tissue, targeted sites in a nerve cell bundle, and targeted sites within nerve cells), while reducing systemic losses into the circulation and reducing collateral tissue or organ damage.

Balloons 410, 510, and 610 may be positioning component which help to hold delivery catheters 400, 500, and 600 in place and assist with the advancement of delivery needles 450, 550, and 650 through the vessel wall W to nerve cell bundles in the adventitia. Balloons 410, 510, and 610 may be made of compliant materials such as nylon or polyurethane. Balloons 410, 510, and 610 may expand at very low pressures, such as approximately 1-2 atmospheres, to prevent injury to the vessel wall W.

Delivery catheters 400, 500, and 600 may be configured to provide blood perfusion during the procedure. The size, number, and shape of needle housings 440 and 540, and needle supports 640, may be configured so that balloons 410, 510, and 610 do not contact the vessel wall W, and vessel wall contact is limited to needle housings 440 and 540, and needle supports 640, only. Balloons 410, 510, and 610 position delivery catheters 400, 500, and 600, assists in conforming needle housings 440, 540, and 640 to the vessel wall W, and helps advance delivery needles 450, 550, and 650 to the targeted sites.

Delivery needles 450, 550, and 650 may be made of Nitinol, stainless steel, or Elgiloy for sufficient stiffness and strength to penetrate the vessel wall W. Delivery needles 450, 550, and 650 may be coated with radiopaque coatings of gold, platinum or platinum-iridium alloy, tantalum, or tungsten to improve the visibility and visualize the advancement of delivery needles 450, 550, and 650 under fluoroscopy.

Delivery needles 450, 550, and 650 may be made of magnetic materials with a very high magnetic permeability such that they are responsive to an external stimulus in a magnetic field. Examples of magnetic materials include, carbon steels, nickel and cobalt-based alloys, Alnico (a combination of aluminum, nickel and cobalt), Hyperco alloy, neodymium-iron boron and samarium-cobalt. Delivery needles 450, 550, and 650 may be advanced into the vessel wall W in a magnetic field using external computer-controlled console systems, such as those manufactured by Stereotaxis. Externally guided ultrasound systems using sound waves traveling through blood may be used to assist with the precise penetration of delivery needles 450, 550, and 650 into the vessel wall W. Delivery needles 450, 550, and 650 may be operated using intravascular microelectromechanical systems (MEMS) that may advance delivery needles 450, 550, and 650 into the vessel wall W using external and/or internal guidance.

Other imaging modalities may be integrated into delivery catheters 400, 500, and 600 to precisely locate target regions inside the body and locally deliver agents within the vessel wall W. These include intravascular ultrasound (IVUS) and optical coherence tomography (OCT) imaging, both of which, have capabilities to distinguish the different layers of the vessel wall (endothelium, intima, media and adventitia). Miniaturized IVUS and OCT sensors can be embedded along the shaft of delivery catheters 400, 500, and 600 and used to track the advancement of delivery needles 450, 550, and 650 into the adventitia. IVUS sensors send sound waves in the 20-40 MHz frequency range; the reflected sound waves from the vessel wall are received through an external computerized ultrasound equipment which reconstructs and displays a real-time ultrasound image of the blood vessel surrounding the sensor. Similarly, OCT sensors produce real-time, high resolution images of the vessel wall (on the order of microns) on computer displays using interferometric methods employing near-infrared light. Both sensors may be located on delivery catheters 400, 500, and 600 near needle ports 446 and 546 at the proximal, middle, or distal segments of balloons 410, 510, and 610. Once the position of delivery needles 450, 550, and 650 is verified, the agent is delivered and delivery needles 450 and 550 retracted.

### VI. METHODS

The nerve affecting compositions and agents may be locally delivered proximate the nerves in the sympathetic nervous system to treat hypertension and other diseases of the autonomic nervous system. As previously stated, the nerve affecting agents may be combined to form a single composition and then injected using the devices described herein. The nerve affecting agents may also be delivered (injected) separately using the same or different devices. The nerve affecting agents may further be delivered to the target tissue simultaneously or sequentially. The target tissue may be a tissue layer of the vascular wall, e.g., the adventitia, or it may be a tissue within the extravascular space. In some variations, the nerve affecting compositions and agents are delivered in a manner so that the pattern of injection follows the curved, winding, or helical/spiral course of the renal nerve on the renal vasculature.

The nerve affecting agents may be provided at or lower than their FDA-approved doses, oral or intravenous. It may be helpful to employ the nerve affecting agents separately or together, in a nerve affecting composition in ratios previously described. For example, if the nerve affecting composition includes digoxin and captopril, the digoxin may comprise 75% (w/v) and captopril 25% (w/v) of the composition. If the nerve affecting composition includes digoxin, phenytoin, and captopril, the digoxin may comprise 50% (w/v), phenytoin 25% (w/v), and captopril 25% (w/v) of the composition. If the nerve affecting composition includes digoxin, chloroquin, verapamil, and lithium chloride, the digoxin may comprise 30% (w/v), chloroquin 30% (w/v), verapamil 20% (w/v), and lithium chloride 20% w/v) of the composition.

The nerve affecting compositions and agents may be delivered to target tissues using the devices described herein. In general, the delivery method includes advancing, e.g., percutaneously, a delivery catheter within a blood vessel and positioning a balloon at or near one or more target sites. Positioning of the balloon at a target site may be aided by the infusion of a radiopaque fluid or contrast agent into the balloon. The radiopaque fluid or contrast agent is typically contained within the balloon and not delivered into the blood vessel lumen, blood vessel wall, or extravascular tissues. In some variations, radiopaque markers disposed at or near the exit openings in the needle housing may be provided. The balloon may then be expanded to bring needle housings in contact with the walls of the vessel. The slideable delivery needles may then be advanced out of needle housings and into the vessel wall. Injection of a nerve affecting composition or agent may thereafter take place. After the composition or agent(s) delivery is complete, the needles may be retracted back into needle housings and the balloon deflated. In some variations, a sheath is employed to deploy the needles instead of sliding them out of needle housings. Here the sheath covers the needles to maintain them in an undeployed state. Upon retraction of the sheath, the needles change configuration to their deployed state and the balloon expanded to force the needles into the wall of the blood vessel. After delivery of the nerve affecting composition or agent(s) is complete, the balloon may be deflated and the sheath advanced over the needles.

The disclosure given above describes how affecting the function of nerves surrounding the renal arteries by the delivery of nerve affecting compositions proximate the nerves may control hypertension. Specifically, delivery of a nerve affecting composition including a cardiac glycoside (e.g., digoxin), a calcium channel blocker (e.g., captopril), and a NSAID (e.g., indomethacin), may affect renal nerve function, and thus, hypertension. However, the described devices, methods, and compositions and agents, may be used to treat other diseases thought to result from dysfunction at various locations along the sympathetic nervous system in the human body. These include and are not limited to diabetes, tingling, tinnitus, fibromyalgia, impulse-control disorders, sleep disorders, pain disorders, pain management, congestive heart failure, sleep apnea, chronic kidney disease and other renal diseases, and obesity. Other potential target sites and disease states are listed below.

| | Target location in the |
|---|---|
| Disease state or condition | sympathetic nervous system |
| Pulmonary hypertension, | Vagus nerve |
| arrhythmias, chronic hunger, | |
| cyclic vomiting syndrome | |
| Pancreatitis, hepatitis, | Celiac ganglia (renal and |
| chronic kidney disease | adrenal nerves etc.) |
| Adrenal function, hypertension | Celiac ganglia, greater |
| | splanchnic nerve |
| Bladder incontinence | Pelvic nerve |
| Hypertension, glaucoma | Carotid artery and plexus |
| Sciatica | Sciatic nerve |
| Fibromyalgia, chicken pox, | Dorsal root ganglia |
| shingles | |
| Mood alteration | Vagus nerve, submaxillary, and |
| | sphenopalatine ganglia |

Methods for treating a disease condition of the autonomic nervous system in a patient are also described that generally include delivering a nerve affecting composition to a portion of a targeted nerve in an amount that affects function of the targeted nerve and alleviates one or more symptoms of the disease condition in the patient, where the nerve affecting composition comprises one or more nerve affecting agents. Here, when the condition is hypertension, the symptoms may include high blood pressure. When the condition is diabetes, the symptoms may include elevated insulin levels, poor glucose tolerance, and/or poor insulin sensitivity. When the condition is renal disease, the symptoms may include poor glomerular filtration rate (GFR). When the condition is obesity, the symptoms may include uncontrolled weight gain. When the condition is atrial fibrillation, the symptoms may include heart palpitations, dizziness, lack of energy and chest discomfort.

Other conditions of the autonomic nervous system, some of which are repeated from above, include depression, fibromyalgia, dementia, attention deficit hyperactivity disorder, sleep apnea, or migraine headaches, and the symptoms include decreased attention, discomfort and overstimulation, congestive heart failure, and the symptoms include shortness of breath, leg swelling, and the inability of the heart to pump sufficient blood into the circulatory system.

### VII. EXAMPLES

### Example 1

### Effect of Nerve Affecting Compositions on Nerve Function

The efficacy of various agents in affecting nerve function was evaluated using a rat sciatic nerve block model. Here rat groups were injected with 0.3 cc of a nerve affecting composition in the left leg near the sciatic notch. The rat groups, compositions, and doses are listed in the table below:

| GROUP | AGENT | DOSE (mg/kg) |
|---|---|---|
| 1 | Ethanol | 100% |
| 2 | Guanethidine | 5.77 |
| 3 | Digoxin | 1.06 |
| 4 | Carbamazepine | 1.44 |
| 5 | Phenytoin | 3.82 |
| 6 | Digoxin + carbamazepine | 0.27 (digoxin), |
| | | 0.36 (carbamazepine) |
| 7 | Digoxin + captopril + indomethacin | 0.27 (digoxin), |
| | | 5.88 (captopril), |
| | | 0.22 (indomethacin) |

FIGS. 12A-12D show the results of the different agents on the rat leg muscles. The effect of the agents was measured based on four tests: (1) nerve conductance, (2) sensory ability, (3) motor function, and (4) pressure exerted.

FIG. 12A shows the results of the nerve conductance test. The nerve conductance test evaluates the ability of electrical current to travel from one electrode, down the sciatic nerve and to a second electrode to form a complete electrical circuit. Nerve conductance was evaluated at two frequencies (1-10 Hz to stimulate leg twitch and 50-100 Hz to stimulate leg tetanus). Impairment in nerve conductance was evaluated at 1, 2, 3, 7, 14, 21, and 30 days post-injection of the compositions. The y-axis scale represents the severity of block (on a scale of 0-3, with 0=no block, 1=slight block, 2=moderate block, 3=severe block).

FIG. 12B shows the results of the sensory ability test. The sensory ability test evaluates sensory nerve function. Needle-nosed forceps were used to pinch the footpad of rat hindlimbs to test ability of sensory nociception. Vocal responses or mechanical withdraw of the foot from the forceps were monitored as pressure increased. Rats were assessed at 1, 2, 3, 7, 14, 21, and 30 days. The y-axis scale represents the severity of sensory nociception block (on a scale of 0-3, with 0=no block, 1=slight block, 2=moderate block, 3=severe block).

FIG. 12C shows the results of the motor function test. The motor function test evaluated the ability of rats to step up, walk, and coordinate their hindlimbs. The measurements were made at 1, 2, 3, 7, 14, 21, and 30 days. The y-axis scale represents the severity of neuromuscular block (on a scale of 0-3, with 0=no block, 1=slight block, 2=moderate block, 3=severe block).

FIG. 12D shows the results of the pressure exerted test. The pressure exerted test evaluated the ability of rats to apply pressure or bear weight on a flat surface which was measured by a digital weighing scale. The measurements were made at 1, 2, 3, 7, 14, 21, and 30 days. The y-axis scale represents the impairment in the ability to bear weight (on a scale of 0-3, with 0=no impairment, 1=slight impairment, 2=moderate impairment, 3=severe impairment).

These data suggest cardiac glycosides, either alone or in combination with an ACE inhibitor and NSAID, outperform guanethidine in the ability to affect peripheral nerve function. Additionally, cardiac glycosides outperform other tested agents, including ethanol, in the ability to impair sensory nociception.

A lower amount of digoxin is needed to affect nerve function when used in conjunction with captopril and indomethacin than when used alone. This synergistic effect may be due to the effect of the captopril and the indomethacin within the same nerve cell, on the neighboring cells, or in the local micro-environment surrounding the nerve cells, nerve cell bundle, or nerve cell junction. For example, co-administration of captopril may have the effect of inhibiting angiotensin II production and reducing nerve stimulation, resulting in decreased nerve activity (e.g., norepinephrine production) in the injected tissue. Additionally, co-administration of indomethacin may have blocked COX-2 activity and prostaglandin production, and therefore decreased healing, which prolonged the effects of digoxin and captopril. Again, the effect of digoxin on nerve cells has not been previously known.

Separate agents for affecting nerve function may be administered using different routes. For digoxin, captopril, and indomethacin, the digoxin may be administered locally in a site-specific manner, while the captopril and the indomethacin may be administered orally or intravenously. The synergistic effects may still be seen, as the combined effects of three separate mechanisms affecting nerve function appear to require smaller doses or local concentrations of each component.

FIG. 13A shows histology from the hind leg of a rat injected with digoxin at 72 hours. The nerve bundles (9000) contain nerve axons showing signs of edema and axonal degeneration. The nerve bundles are surrounded by perineuritis (9001).

FIG. 13B shows histology from the hind leg of a rat injected with digoxin at 30 days. The nerve bundles (9002) contain degenerated nerves. The absence of inflammatory foci surrounding the degenerative nerve bundles is also noted (9003).

The following table is a summary of the effects of three different compositions on the nerve cells.

| | Time | Sciatic Nerve | Inflammatory |
|---|---|---|---|
| Agent | Point | Pathology Report | Condition |
| Phenytoin | 72 hrs | Normal | Normal |
| | 30 days | Normal | Perineuritis |
| Digoxin | 72 hrs | Normal | Perineuritis |
| | 30 days | Degenerative with some | No inflammation |
| | | edema; endoneurium is | |
| | | absent; nerve is frag- | |
| | | mented; axonal degener- | |
| | | ation is present | |
| Digoxin + | 72 hrs | Nerve degeneration with | No inflammation |
| captopril + | | edema | |
| indomethacin | | | |
| | 30 days | Axonal degeneration with | No inflammation |
| | | some swelling; no hyper- | |
| | | cellularity | |

1. A method for treating hypertension in a patient, the method comprising:
delivering a cardiac glycoside locally to a portion of a renal nerve in an amount that affects function of the renal nerve and lowers blood pressure of the patient.

2. The method of claim 1, wherein the amount of the cardiac glycoside delivered reduces nerve conductance in the portion of the renal nerve.

3. The method of claim 1, wherein the amount of the cardiac glycoside delivered induces death of nerve cells in the portion of the renal nerve and prevents regrowth of nerve cells.

4. The method of claim 1, wherein the amount of the cardiac glycoside delivered affects nerve function by inducing neuro-muscular block, sensory nerve block, or clinical nerve block.

5. The method of claim 1, wherein the amount of the cardiac glycoside delivered is approximately 0.05-1 mg/kg.

6. The method of claim 1, wherein the volume of the cardiac glycoside delivered is approximately 0.05-5 ml per administration.

7. The method of claim 1, wherein the portion of the renal nerve constitutes the axonal segment.

8. The method of claim 1, wherein the portion of the renal nerve constitutes receptors that receive signals from cells that can activate nerves.

9. A method for treating hypertension in a patient, the method comprising:
locally delivering a nerve affecting composition to a portion of a renal nerve in an amount that affects function of the renal nerve and lowers blood pressure of the patient, wherein the nerve affecting composition comprises a cardiac glycoside, an ACE inhibitor, and an NSAID.

10. The method of claim 9, wherein the amount of the composition delivered reduces nerve conductance in the portion of the renal nerve.

11. The method of claim 9, wherein the amount of the composition delivered induces death of nerve cells in the portion of the renal nerve.

12. The method of claim 9, wherein the amount of the composition delivered induces death of nerve cells in the portion of the renal nerve and prevents regrowth of nerve cells.

13. The method of claim 9, wherein function of the renal nerve is affected temporarily.

14. The method of claim 9, wherein function of the renal nerve is affected for a sustained period of time.

15. The method of claim 9, wherein the composition is delivered in a time release formulation.

16. The method of claim 9, wherein the cardiac glycoside comprises digoxin.

17. The method of claim 9, wherein the ACE inhibitor comprises captopril.

18. The method of claim 9, wherein the non-steroidal anti-inflammatory comprises indomethacin.

19. The method of claim 9, wherein the amount of the composition delivered is approximately 0.2-2 mg/kg of the cardiac glycoside, approximately 2-20 mg/kg of the ACE inhibitor, and approximately 0.2-2 mg/kg of the NSAID.

20. A method for treating a disease condition of the autonomic nervous system in a patient, the method comprising:
delivering a nerve affecting composition to a portion of a targeted nerve in an amount that affects function of the targeted nerve and alleviates one or more symptoms of the disease condition in the patient,
wherein the nerve affecting composition comprises one or more nerve affecting agents.

21. The method of claim 20, wherein the condition is hypertension, and the symptoms include high blood pressure.

22. The method of claim 20, wherein the condition is diabetes, and the symptoms include elevated insulin levels, poor glucose tolerance, and poor insulin sensitivity.

23. The method of claim 20, wherein the condition is renal disease, and the symptoms include poor glomerular filtration rate (GFR).

24. The method of claim 20, wherein the condition is depression, fibromyalgia, dementia, attention deficit hyperactivity disorder, sleep apnea, or migraine headaches, and the symptoms include decreased attention, discomfort and overstimulation, congestive heart failure, and the symptoms include shortness of breath, leg swelling, and the inability of the heart to pump sufficient blood into the circulatory system.

25. The method of claim 20, wherein the condition is obesity, and the symptoms include uncontrolled weight gain.

26. The method of claim 20, wherein the condition is atrial fibrillation, and the symptoms include heart palpitations, dizziness, lack of energy and chest discomfort.

27. The method of claim 20, wherein the nerve affecting agent comprises a cardiac glycoside.

28. The method of claim 27, wherein the cardiac glycoside comprises digoxin.

29. The method of claim 20, wherein the nerve affecting agent comprises an ion channel blocker.

30. The method of claim 29, wherein the ion channel blocker comprises phenytoin.

31. The method of claim 29, wherein the ion channel blocker comprises carbamazepine or lithium chloride.

32. The method of claim 20, wherein the nerve affecting agent comprises an ACE inhibitor.

33. The method of claim 20, wherein the nerve affecting agent comprises an antibiotic.

34. The method of claim 20, wherein the nerve affecting agent comprises an excitatory glutamate receptor.

35. A method for treating a disease condition of the autonomic nervous system in a patient, the method comprising:
delivering a nerve affecting composition to a portion of a targeted nerve in an amount that affects function of the targeted nerve and alleviates one or more symptoms of the disease condition in the patient, wherein the nerve affecting composition comprises a cardiac glycoside, an ACE inhibitor, and an NSAID.

36. The method of claim 35, wherein the nerve affecting agent is delivered locally.

37. The method of claim 35, wherein the nerve affecting agent is delivered orally.

38. The method of claim 35, wherein the targeted nerve is affected by temporary neuromuscular block, sustained neuromuscular block, sensory nerve block, or clinical nerve block.

39. The method of claim 35, wherein the targeted nerve is affected by reduced or blocked nerve conductance.

40. The method of claim 35, wherein the targeted nerve is affected by nerve cell death.

41. The method of claim 35, wherein the targeted nerve is affected by damage to axonal segments of neurons.

42. The method of claim 35, wherein the nerve affecting agents are selected from one or more of the following: agents which inhibit sodium-potassium pumps, calcium channels and sodium channels in nerve cells; angiotensin converting enzymes;
glutamate receptors; COX-1 and COX-2 receptors in nerve cells.

43. The method of claim 35, wherein the amount of agent delivered is sufficient to impair nerve function by acting on Schwann cells.

44. A delivery catheter comprising:
a balloon having a proximal portion and a distal portion;
a proximal cap coupled to the proximal portion of the balloon;
a distal cap slidably coupled to the distal portion of the balloon;
a plurality of needle housings having proximal portions and distal portions, the proximal portions of the needle housings being coupled to the proximal cap, the distal portions of the needle housings being coupled to the distal cap; and
a delivery needle slidably disposed within a needle lumen formed in each of the needle housings, the delivery needles capable of being advanced and retracted through a needle port formed in an outwardly-facing side of each needle housing.

45. The device of claim 44, wherein the needle housing has a substantially helical configuration.

46. The device of claim 44, where the device profile is between 4-8F.

47. The device of claim 44, where the device has exceptional conformability and torquability while delivering therapy in a tortuous anatomy.

48. The device of claim 44, wherein the delivery needles are coated for improved visibility under various imaging modalities including ultrasound, X-rays, OCT and MRI.

49. The device of claim 44, wherein the delivery needles are coated with a sealing agent to promote sealing of the vessel wall upon needle retraction after delivering the agent.

50. The device of claim 44, wherein the delivery needles are coated with a anti-inflammatory compound to promote healing of the vessel wall upon needle retraction after delivering the agent.

51. The device of claim 44, where the proximal (handle) end of the needle assembly is equipped with a pressure or force sensor to monitor contact with the vessel wall and subsequent advancement of the needle into the vessel wall.

52. The device of claim 44, where the proximal (handle) end of the needle assembly is equipped with a gage to monitor depth of penetration into the vessel wall.

53. The device of claim 44, where the proximal (handle) end of the needle assembly is equipped with a mechanical stop to limit the maximum depth of penetration into the vessel wall.

54. The device of claim 44, where the needle housings and needle exit ports are equipped with radiopaque markers to assist viability under fluoroscopy.

55. A delivery catheter comprising:
a balloon having a proximal portion and a distal portion;
a proximal cap coupled to the proximal portion of the balloon;
a distal cap coupled to the distal portion of the balloon;
a plurality of needle housings having proximal portions and distal portions, the proximal portions of the needle housings being coupled to the proximal cap, the distal portions of the needle housings being slidably disposed within one or more openings in the distal cap; and
a delivery needle slidably disposed within a needle lumen formed in each of the needle housings, the delivery needles capable of being advanced and retracted through a needle port formed in an outwardly-facing side of each needle housing.

56. A delivery catheter comprising:
a balloon having a proximal portion and a distal portion;
a proximal cap coupled to the proximal portion of the balloon;
a distal cap coupled to the distal portion of the balloon;
a plurality of needle supports having proximal portions and distal portions, the proximal portions of the needle supports being coupled to the proximal cap, the distal portions of the needle supports being coupled to the distal cap, each of the needle supports having a delivery lumen;
a delivery needle coupled to each needle support, the delivery needles being outwardly biased, each of the delivery needles having a delivery lumen in fluid communication with the delivery lumen of each needle support; and
a sheath slidably coupled around the delivery needles, the sheath capable of constraining the delivery needles.
